(19) United States
(12) Patent Application Publication
(10) Pub. No.: US 2024/0345083 A1
(43) Pub. Date: Oct. 17, 2024

(54) METHODS FOR OBTAINING ANTIBODY MOLECULES WITH HIGH AFFINITY

(71) Applicant: Regeneron Pharmaceuticals, Inc., Tarrytown, NY (US)

(72) Inventors: Wen-Yi Lee, New Hyde Park, NY (US); Gang Chen, Yorktown Heights, NY (US); Kristel Velez, Flushing, NY (US); George D. Yancopoulos, Yorktown Heights, NY (US)

(73) Assignee: Regeneron Pharmaceuticals, Inc., Tarrytown, NY (US)

(21) Appl. No.: 18/604,573

(22) Filed: Mar. 14, 2024

Related U.S. Application Data
(60) Provisional application No. 63/452,206, filed on Mar. 15, 2023.
(60) Provisional application No. 63/559,544, filed on Feb. 29, 2024.

(51) Int. Cl. G01N 33/569; C07K 16/10; C07K 16/24

(52) U.S. Cl. CPC G01N 33/56972; C07K 16/10; C07K 16/244; C07K 2317/92

(57) ABSTRACT

Disclosed herein are methods for obtaining antibody-producing cells that express antibody molecules exhibiting high binding affinity for an antigen which comprise contacting a population of antibody-producing cells encompassing cells that express antibody molecules to the antigen on the cell surface, with a first labeled form of the antigen to allow the antigen to bind to the antibody molecules on the cell surface, followed by contacting the cells with (i) an unlabeled form of the antigen, (ii) a second labeled form of the antigen, or (iii) the unlabeled form of the antigen and the second labeled form of the antigen; and collecting cells that remain bound to the first labeled form of the antigen, thereby obtaining cells expressing high affinity antibody molecules to the antigen. The present methods allow for obtaining cells expressing high affinity antibody molecules from a pool of antibody-producing cells that express antibody molecules with different affinities.

### CROSS REFERENCE TO RELATED APPLICATIONS

This application claims the benefits of priority from U.S. Provisional Applications 63/452,206, filed Mar. 15, 2023 and 63/559,544, filed Feb. 29, 2024, the contents of both of which are incorporated herein by reference.

### BACKGROUND

High affinity binding is a desirable attribute for many therapeutic antibody molecules. The availability of high affinity monoclonal antibodies is crucial to the development of targeted immunotherapies. In many immunized animals, however, very high affinity antibody molecules are rare and standard methods for generating antibody molecules are inefficient at isolating high affinity antibody molecules.

Typically, monoclonal antibodies are obtained from mouse hybridomas, most often resulting from the fusion of B lymphocytes from immunized mice with murine myeloma cells. The isolation of rare high affinity antibodies by hybridoma technology, however, is not efficient because of throughput limitations on hybridoma culture.

Another approach to producing high affinity antibody molecules involves the use of display technology to produce a lead antibody candidate from a phage, yeast or mammalian library. Though direct DNA isolation from B cells expressing antibody molecules may be utilized, DNA libraries are expressed in cell expression systems, such as phage, yeast, or bacterial systems, then “panned” or titrated to select for the antibody molecules having high affinities. Display technologies can provide high-quality protein libraries, although they provide limited diversity. Consequently, in vitro mutagenesis-based affinity maturation is frequently a next step in generating high affinity antibody molecules derived from such libraries.

Thus, a need exists for an efficient method to obtain, in quantity, antibody molecules that have the requisite specificity and high binding affinity in an efficient manner, without the need for several rounds of screening (such as “panning”) or site-directed mutagenesis.

### SUMMARY

Disclosed herein are methods for obtaining B cells and other antibody-producing cells that express antibody molecules exhibiting high binding affinity for an antigen as well as a mammalian host cell made by the methods disclosed. The present disclosure is based on the observation that cells expressing antibody molecules with high affinity for an antigen of interest can be selected and enriched directly from a population of antibody-producing cells of varying affinities. The antibody molecules are thus selected directly from B cells and other antibody-producing cells, prior to a single-cell isolation and collection, such as using fluorescence activated cell sorting (FACS). This provides a simple method for obtaining cells expressing high affinity antibody molecules from a pool of B cells and other antibody-producing cells that express antibody molecules with different affinities.

In one aspect, the present disclosure is directed to a method for obtaining antibody-producing cells that express antibody molecules exhibiting high binding affinity for an antigen, the method comprising:
(a) contacting a population of antibody-producing cells that express antibody molecules to the antigen on the cell surface, with a first labeled form of the antigen to allow the antigen to bind to the antibody molecule on the cell surface, wherein the antigen of the first labeled form is conjugated to a first detectable label;
(b) washing the cells to remove unbound antigen;
(c) contacting the cells with
   (i) an unlabeled form of the antigen,
   (ii) a second labeled form of the antigen, or
   (iii) the unlabeled form of the antigen and the second labeled form of the antigen;
(d) washing the cells to remove unbound antigen; and
(e) collecting cells that remain bound to the first labeled form of the antigen, thereby obtaining cells expressing high affinity antibody molecules to the antigen.

In some embodiments of the method, the first labeled form of the antigen is at a concentration between 0.001 nM and 1 μM. In some embodiments of the method, the first labeled form of the antigen is at a concentration between 0.05 nM and 10 nM. In some embodiments, the first labeled form of the antigen is at a concentration between 0.1 and 7.5 nM. In some embodiments, the first labeled form of the antigen is at a concentration of about 0.2 nM. In some embodiments, the first labeled form of the antigen is at a concentration of about 5 nM.

In some embodiments, the first detectable label is a first fluorescent label.

In some embodiments, the antigen is a protein in a monomeric form. In some embodiments, the antigen is a protein in a multimeric form, e.g., dimer, trimer, tetramer, pentamer, hexamer, among others, or a mixture thereof. In some embodiments, the antigen is a protein that is present in both monomeric and multimeric forms.

In some embodiments, the first labeled form of the antigen is a monovalent form of the antigen. In such embodiments, the antigen can be a protein in a monomeric form, a protein in a multimeric form, or a protein in both monomeric and multimeric forms, and there is no multimerization of the antigen in a monovalent form. In some embodiments, the first labeled form of the antigen is a multivalent form of the antigen. In such embodiments, the antigen can be a protein in a monomeric form, a protein in a multimeric form, or a protein in both monomeric and multimeric forms, and multiple units of the antigen is presented in a multivalent form. In some embodiments, the first labeled form of the antigen is a mixture of monovalent and multivalent forms of the antigen. In embodiments where a multivalent form of the antigen is employed, the multivalent form of the antigen can be provided by a multivalent molecule to which the antigen is bound or linked. In some embodiments, the multivalent molecule is a streptavidin multimer (e.g., tetramer), which can be bound by biotin to which the antigen is conjugated. In some embodiments, the multivalent molecule is a dimer of an immunoglobulin Fc fragment. In some embodiments, the multivalent molecule is a trimer of a trimerization domain molecule such as foldon.

In some embodiments, an unlabeled form of the antigen, which can be employed in the chase step, is a monovalent form of the antigen, wherein the antigen can be a protein in a monomeric form, a protein in a multimeric form, or a protein in both monomeric and multimeric forms. In some embodiments, the unlabeled form of the antigen is a multivalent form of the antigen, wherein the antigen can be a protein in a monomeric form, a protein in a multimeric form, or a protein in both monomeric and multimeric forms. In some embodiments, the unlabeled form of the antigen comprises a mixture of a monovalent form and a multivalent form of the antigen, wherein the antigen can be a protein in a monomeric form, a protein in a multimeric form, or a protein in both monomeric and multimeric forms. In embodiments where a multivalent form of the antigen is employed, the multivalent form of the antigen can be provided by a multivalent molecule to which the antigen is bound or linked. In some embodiments, the multivalent molecule is a streptavidin multimer (e.g., tetramer), which can be bound by biotin to which the antigen is conjugated. In some embodiments, the multivalent molecule is a dimer of an immunoglobulin Fc fragment. In some embodiments, the multivalent molecule is trimer of a trimerization domain molecule such as foldon.

In some embodiments, a second labeled form of the antigen, which can be employed in the chase step, is a monovalent form of the antigen, wherein the antigen can be a protein in a monomeric form, a protein in a multimeric form, or a protein in both monomeric and multimeric forms. In some embodiments, the second labeled form of the antigen is a multivalent form, wherein the antigen can be a protein in a monomeric form, a protein in a multimeric form, or a protein in both monomeric and multimeric forms. In some embodiments, the second labeled form of the antigen comprises a mixture of a monovalent form and a multivalent form, wherein the antigen can be a protein in a monomeric form, a protein in a multimeric form, or a protein in both monomeric and multimeric forms. In embodiments where a multivalent form of the antigen is employed, the multivalent form of the antigen can be provided by a multivalent molecule to which the antigen is bound or linked. In some embodiments, the multivalent molecule is a streptavidin multimer (e.g., tetramer), which can be bound by biotin to which the antigen is conjugated. In some embodiments, the multivalent molecule is a dimer of an immunoglobulin Fc fragment. In some embodiments, the multivalent molecule is trimer of a trimerization domain molecule such as foldon. The second labeled form of the antigen is labeled with a second detectable label, which is different from the first detectable label. In some embodiments, the second detectable label is a second fluorescent label, which is different from the first fluorescent label if used.

In embodiments where an unlabeled form of the antigen is used in the chase, the unlabeled form can be a monovalent form of the antigen, a multivalent form of the antigen, or a mixture thereof, wherein the antigen can be a protein in a monomeric form, a protein in a multimeric form, or a protein in both monomeric and multimeric forms. In some embodiments, the unlabeled form of the antigen is a monovalent form of the antigen. In some embodiments, the antigen in the unlabeled form is at least 2 to 150-fold and up to, e.g., 2500 fold in molar ratio relative to the first labeled form of the antigen. For example, the antigen in the unlabeled form is at least 2 to at least 120-fold, including 2 fold, 3 fold, 4 fold, 5 fold, 10 fold, 15 fold, 20 fold, 25 fold, 50 fold, 75 fold, 100 fold, 125 fold, or 150 fold, up to, e.g., 2500 fold, in molar ratio relative to the first labeled form of the antigen. In some embodiments, depending on the concentration of the first labeled form of the antigen, the unlabeled form of the antigen in the chase step can be at a concentration between 0.4 nM to 600 nM.

In embodiments wherein a second labeled form of the antigen is used in the chase, the second labeled form of the antigen can be a monovalent form, a multivalent form, or a mixture thereof, a monomeric form and multimeric form, wherein the antigen can be a protein in a monomeric form, a protein in a multimeric form, or a protein in both monomeric and multimeric forms. In some embodiments, the second labeled form of the antigen is a multivalent form of the antigen. In some embodiments, the antigen in the second labeled form is at least 2 to 150-fold and up to, e.g., 2500 fold in molar ratio relative to the first labeled form of the antigen. For example, the antigen in the second labeled form is at least 2 to at least 120-fold, including 2 fold, 3 fold, 4 fold, 5 fold, 10 fold, 15 fold, 20 fold, 25 fold, 50 fold, 75 fold, 100 fold, 125 fold, or 150 fold, up to e.g., 2500 fold in molar ratio relative to the first labeled form of the antigen. In some embodiments, depending on the concentration of the first labeled form of the antigen, the second labeled form of the antigen is at a concentration between 15 to 600 nM.

In some embodiments, the chase step and the wash after the chase step are repeated at least once before collecting cells that remain bound to the first labeled form of the antigen.

In embodiments wherein an unlabeled form of the antigen and a second labeled form of the antigen are used in the chase, the unlabeled form can be a monovalent form, a multivalent form, or a mixture of a monovalent and multivalent form of the antigen, and the second labeled form of the antigen can be a monovalent form, a multivalent form, or a mixture of a monovalent and multivalent form of the antigen. In some embodiments, the chase is performed with an unlabeled, monovalent form of the antigen and a second labeled form of the antigen that is multivalent. In some embodiments of a combination chase, the unlabeled form of the antigen is at least 2-fold to 150-fold and up to e.g., 2500 fold in molar ratio relative to the first labeled form of the antigen, e.g., at least 2 to at least 150-fold, including 2 fold, 3 fold, 4 fold, 5 fold, 10 fold, 15 fold, 20 fold, 25 fold, 50 fold, 75 fold, 100 fold, 125 fold, or 150 fold, and up to, e.g., 2500 fold; and the second labeled form of the antigen is at least 2-fold to 150-fold and up to e.g., 2500 fold in molar ratio relative to the first labeled form of the antigen, e.g., at least 2 to at least 150-fold, including 2 fold, 3 fold, 4 fold, 5 fold, 10 fold, 15 fold, 20 fold, 25 fold, 50 fold, 75 fold, 100 fold, 125 fold, or 150 fold, and up to, e.g., 2500 fold.

In some embodiments, the first detectable label is a fluorescent label, and fluorescence-activated cell sorting is used to collect cells that remain bound to the first labeled form of the antigen. In some embodiments, the first detectable label is a first fluorescent label, the second detectable label is a second fluorescent label that differentiates from the first fluorescent label, and two-dimensional fluorescence-activated cell sorting is used to collect cells that remain bound to the first labeled form of the antigen.

In some embodiments, the antibody producing cells are mammalian cells or yeast (such as S. cerevisiae or Pichia) that produce antibody molecules on the cell surface. In some embodiments, the antibody producing mammalian cells are primary antibody producing cells or immortalized cells lines which produce antibody molecules on the cell surface. In some embodiments, the primary antibody-producing cells are obtained from the spleen, lymph node, peripheral blood and/or bone marrow of a mammalian subject (e.g., human, rabbit, pig, cow, horse, and rodent). In some embodiments, the immortalized cells lines are chosen from Chinese hamster ovary (CHO) cells, Human Embryonic Kidney (HEK) 293 cells, and hybridoma cells which produce antibody molecules on the cell surface.

In some embodiments, the cells collected in step (e) are enriched for cells expressing high affinity antibody molecules. In some embodiments, the high affinity of an antibody molecule obtained has a KD of less than 25 nM. In some embodiments, the high affinity of an antibody molecule obtained is in the range of from 0.1 pM to about 25 nM (KD). In some embodiments, the high affinity is less than 10 nM (KD). In some embodiments, the high affinity is less than 5 nM (KD). In some embodiments, the high affinity is less than 1 nM (KD). In some embodiments, the high affinity is less than 0.1 nM (KD). In some embodiments, the high affinity is less than 0.01 nM (KD). In some embodiments, the high affinity is less than 5 pM (KD). In some embodiments, the high affinity is less than 1 pM (KD). In some embodiments, at least 40% (e.g., 40%, 50%, 60%, 70%, 80%, 90% or more) of the cells collected express a high affinity antibody molecule, e.g., antibody molecules having a KD from 0.1 pM to 25 nM. In some embodiments, the frequency of cells expressing high affinity antibody molecules (e.g., antibody molecules having a KD from 0.1 pM to 25 nM) in the cell population after a chase is increased by at least 30% (e.g., 30%, 40%, 50%, 75%, 100%, 200% or more) as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 30% (e.g., 30%, 40%, 50%, 75%, 100%, 200% or more) as compared to the frequency in the cell population before or without the chase. In some embodiments, the frequency of cells expressing high affinity antibody molecules having a KD of less than 0.1 nM (e.g., antibody molecules having a KD from 0.1 pM to 0.1 nM) in the cell population after a chase is increased by at least 30% (e.g., 30%, 40%, 50%, 75%, 100%, 200% or more) as compared to the frequency in the cell population before or without the chase.

In some embodiments, the method further comprises isolating antibody-encoding nucleic acids from the cells collected that remain bound to the first labeled form of the antigen.

In some embodiments, the method further comprises transfecting a host cell with a nucleic acid encoding an antibody heavy chain or variable domain thereof, and a nucleic acid encoding an antibody light chain or variable domain thereof; and growing the transfected cell under conditions to support expression of antibody by the transfected cell. In some embodiments, the host cell is Chinese hamster ovary (CHO) cell.

In another aspect, the disclosure is directed to a mammalian host cell made by isolating antibody-encoding nucleic acids from the collected cells expressing high affinity antibody molecules, and transfecting a host cell with a nucleic acid encoding an antibody heavy chain or variable domain thereof, and a nucleic acid encoding an antibody light chain or variable domain thereof; and growing the transfected host cell under conditions to support expression of antibody molecules by the host cell. In some embodiments, the host cell is a CHO cell.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 is a representation showing the overall view of the methods used in the disclosure. Beads or cells coated with antibodies of varying affinities are exposed and bound to A647 conjugated antigen (i.e., sort agent or a first labeled form of the antigen) at a concentration, e.g., 5 nM or 0.2 nM, before undergoing a chase step. Three exemplary options of chase are illustrated: A. a cold (unlabeled) monovalent chase with unlabeled monovalent antigen at a concentration at least 2-fold and up to 2500 fold relative to the labeled sort antigen; B. a fluorophore labeled (or “hot”) multivalent chase of pre-bound biotin labeled antigen with streptavidin (SA)-Phycoerythrin (PE) (with the antigen at a concentration at least 4-fold and up to 2500 fold relative to the labeled sort antigen, and a 4:1 ratio of biotin labeled antigen to SA-PE); or C. a combination of an unlabeled monovalent antigen at a concentration at least 2 fold and up to 2500 fold relative to the labeled sort antigen, and a hot multivalent agent using a pre-bound biotin labeled antigen with SA-PE (with antigen at a concentration at least 2-fold and up to 2500 fold relative to the labeled sort antigen, and a 4:1 ratio of biotin labeled antigen to SA-PE).

FIG. 2 is a representation showing the overall view of the methods used in the disclosure with human IL-13 as the antigen. Beads coated with IL-13 antibodies of varying affinities were exposed and bound to A647 conjugated human IL13 (i.e. sort agent) at a concentration, e.g., 5 nM or 0.2 nM, before undergoing a chase step. Three exemplary options of chase are illustrated: A. a cold (unlabeled) monovalent chase with unlabeled human IL13 at a concentration at least 4-fold and up to 2500 fold relative to the labeled sort antigen; B. a fluorophore labeled (hot) multivalent chase of pre-bound biotin labeled human IL13 with streptavidin (SA)-Phycoerythrin (PE)-(with human IL13 at a concentration at least 4-fold and up to 2500 fold relative to the labeled sort antigen, and a 4:1 ratio of biotin labeled human IL13 to SA-PE); or C. a combination of an unlabeled monovalent chase agent human IL13 at a concentration at least 4 fold and up to 2500 fold relative to the labeled sort antigen, and a hot multivalent chase agent using a pre-bound biotin labeled human IL13 with SA-PE (with human IL13 at a concentration at least 4-fold and up to 2500 fold relative to the labeled sort antigen, and a 4:1 ratio of biotin labeled human IL13 to SA-PE).

FIG. 3 is a representative univariate histogram overlay of A647 staining of the IL13 antibody coated beads without chase, as detected by flow cytometry. Four different groups of beads were coated with IL13 antibodies having different dissociation constants (as set forth in the insert box) and were exposed to 5 nM A647 conjugated human IL13, followed by two washes. A similar experiment was conducted with 0.2 nM A647 conjugated human IL13 yielding similar results (not shown).

FIG. 4 is a representative univariate histogram overlay of A647 staining of the IL13 antibody coated beads with cold monomeric chase detected by flow cytometry. Four different groups of beads were coated with IL13 antibodies having different dissociation constants (as set forth in the insert box) and were exposed to 5 nM A647 conjugated human IL13, washed, and were further incubated twice with 4× (20 nM) of unlabeled human IL13 for 45 minutes with two washes in-between, before the detection of A647 by flow cytometry. A similar experiment was conducted with the same four different groups of beads coated with IL13 antibodies and exposed to 0.2 nM A647 conjugated human IL13, washed, and further incubated twice with 20 nM (i.e., 10×) of unlabeled human IL13 yielding similar results (not shown).

FIG. 5 is a representative univariate histogram overlay of A647 staining of the IL13 antibody coated beads with cold monovalent chase detected by flow cytometry. Four different IL13 antibody coated beads were exposed to 5 nM A647 conjugated human IL13, washed, and were further incubated twice with 100× (500 nM) of unlabeled human IL13 for 45 minutes with two washes in-between. A similar experiment was conducted exposing the same four different IL13 antibody coated beads to 0.2 nM A647 conjugated human IL13, washed, and further incubated twice with 100× (20 nM) unlabeled human IL13 yielding similar results (not shown).

FIG. 6 is a representative univariate histogram overlay of A647 staining of the IL13 antibody coated beads with fluorophore labeled multivalent chase detected by flow cytometry. Four different groups of beads were coated with IL13 antibodies having different dissociation constants and exposed to 5 nM A647 conjugated human IL13, washed, and further incubated twice with 4× (20 nM) biotinylated human IL13 pre-clustered by 5 nM SA-PE for 45 minutes with two washes in-between. A similar experiment was conducted exposing the four different IL13 antibody coated beads to 0.2 nM A647 conjugated human IL13, washed, and further incubated twice with 100× (20 nM) biotinylated human IL13 pre-clustered by 5 nM SA-PE yielding similar results (not shown).

FIG. 7 is a representative univariate histogram overlay of A647 staining of IL13 antibody coated beads with the combined chase (cold monovalent antigen and hot multivalent antigen). Four different groups of beads were coated with IL13 antibodies having different dissociation constants and were exposed to 5 nM A647 conjugated human IL13, washed, and further incubated twice with 500 nM of human IL13 and 20 nM biotinylated human IL13 pre-clustered by 5 nM SA-PE for 45 minutes with two washes in-between.

FIG. 8 is a representative A647 vs PE dot plot (i.e., the chase map) of IL13 antibody coated beads with fluorophore labeled multivalent chase, detected by flow cytometry. Four different groups of beads coated with IL13 antibodies having different dissociation constants were exposed to 5 nM A647 conjugated human IL13, washed, and then further incubated twice with 4× (20 nM) biotinylated human IL13 pre-clustered by 5 nM SA-PE for 45 minutes with two washes in-between. A similar experiment was conducted exposing the four different IL13 antibody coated beads to 0.2 nM A647 conjugated human IL13, washed, and further incubated twice with 100× (20 nM) biotinylated human IL13 pre-clustered by 5 nM SA-PE yielding similar results (not shown).

FIG. 9 is a representative A647 vs PE dot plot (i.e., chase map) of IL13 antibody coated beads with the combined chase (cold monovalent and hot multivalent), detected by flow cytometry. Four different groups of beads coated with IL13 antibodies having different dissociation constants were exposed to 5 nM A647 conjugated human IL13, washed, and further incubated twice with 100× (500 nM) of human IL13 and 4× (20 nM) biotinylated human IL13 pre-clustered by 5 nM SA-PE for 45 minutes with two washes in-between.

FIG. 10 is a representative A647 vs PE dot plot (i.e., chase map) of IL13 antibody coated beads with fluorophore labeled multivalent chase, detected by flow cytometry. Four different groups of beads coated with IL13 antibodies having different dissociation constants were exposed to 0.2 nM A647 conjugated human IL13, washed, and then further incubated with 100× (20 nM) biotinylated human IL13 pre-clustered by SA-PE for 45 minutes with two washes in-between.

FIG. 11 is a representative A647 vs PE dot plot (i.e., chase map) of IL13 antibody coated beads with the combined chase (cold monovalent and hot multivalent), detected by flow cytometry. Four different groups of beads coated with IL13 antibodies having different dissociation constants were exposed to 0.2 nM A647 conjugated human IL13, washed, and further incubated with 500 nM of human IL13 and 20 nM biotinylated human IL13 pre-clustered by 5 nM SA-PE for 45 minutes with two washes in-between.

FIG. 12A-C shows results of splenocytes from one mouse sorted according to the strategies as described above. A. No chase. B. Hot multivalent chase. C. Combination Chase (Cold monovalent and hot multivalent). All cells were first gated based on viability and IgG expression. The final sort gates on the A647 vs PE dot plot are highlighted in yellow on the flow cytometry profiles, the upper panels. The gated cell populations were index sorted and analyzed based on the Biacore data as shown in the lower portion of the figures. The no-chase sorting strategy (A) yielded the antibodies with the shortest t_{1/2} values whereas the chase strategies enriched for antibodies with longer t_{1/2}s (B and C).

FIG. 13A-C shows results of antibodies from a representative mouse (#6018066) where the antibodies were analyzed by Biacore. A. No chase. B. Hot multivalent chase. C. Combination Chase (cold monovalent and hot multivalent). The antibodies that were isolated without a chase were mostly low affinity with short t_{1/2}s (A). However, the antibodies that were isolated using either of the 2 chase strategies show a marked enrichment for sub-nanomoloar affinity antibodies with t_{1/2}s over 100 minutes (B and C). In addition to having improved affinity properties, the antibodies from the chase conditions were also potent inhibitors in the IL13 inhibition assay (as designated by the color of the data points—yellow is 100% inhibition).

FIG. 14A-F show the antibodies from all the mice that were analyzed by Biacore. The trends seen in the individual mouse example of FIG. 13 were also consistently observed across all the mice. FIGS. 14B, 14D, and 14F are zoomed in views of the boxed inlays and focus on the higher affinity antibodies. A and B: no chase; C and D: hot multivalent chase; E and F: cold monovalent plus hot multivalent chase. Overall, a greater than 3-fold enrichment in longer t_{1/2} antibodies and greater than 2-fold enrichment in the overall K_{D}s were observed. Additionally, the high affinity antibodies from the Cold Monovalent+Hot Multivalent Chase strategy (E and F) also have a propensity for being strong IL13 Bioassay blockers (100-200 pM hIL13 was used in Bioassay).

FIG. 15A-D show dot plot overlays of polystyrene microbeads separately coated with two monoclonal antibodies against Zaire Ebola GP protein with different affinities: anti Ebola GP-1, and anti Ebola GP-2. Antibody coated beads were further treated with either no-chase condition (A and C), or fluorophore labeled multivalent chase condition (B and D). Beads shaded in green (t_{1/2}=1155 min, coated with anti Ebola GP-1) showed less PE staining and more A647 staining, which is separated from beads shaded in purple (t_{1/2}=3 min, coated with anti Ebola GP-2) that has more PE staining and less A647 staining. Beads coated with the two different antibodies can be separated from each other using fluorophore labeled multivalent chase method based on the dissociation rates.

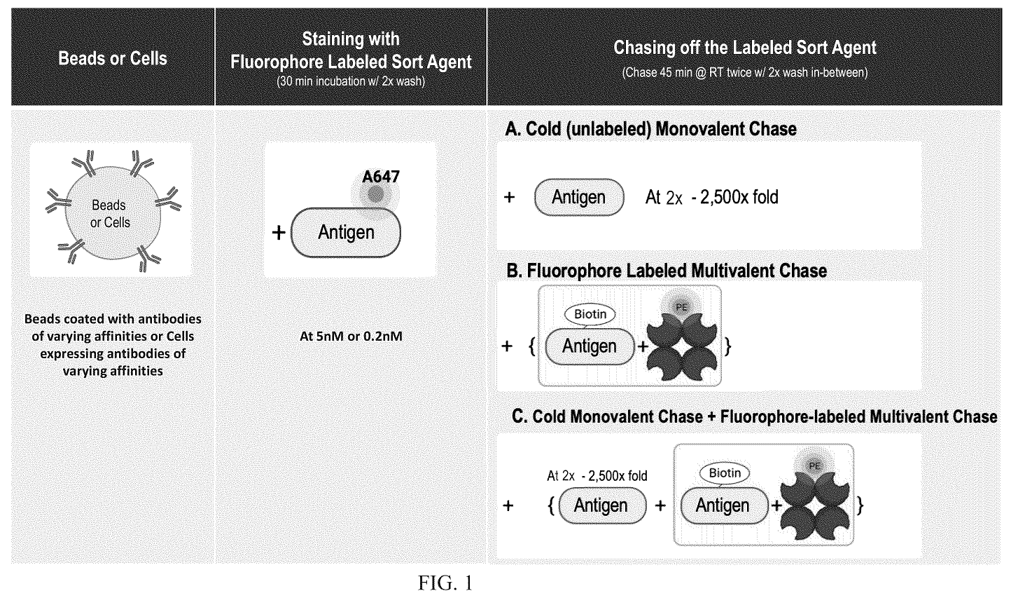

### DETAILED DESCRIPTION

Disclosed herein are methods for obtaining primary antibody-producing cells that express antibodies exhibiting high binding affinity for an antigen.

Although claimed subject matter will be described in terms of certain examples, other examples, including examples that do not provide all the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

In the description that follows, certain conventions will be followed as regards to the usage of terminology. Generally, terms used herein are intended to be interpreted consistently with the meaning of those terms as they are known to those of skill in the art. In practicing the present disclosure, many conventional techniques in molecular biology, microbiology, cell biology, biochemistry, and immunology are used, which are within the skill of the art. These techniques are described in greater detail in, for example, Molecular Cloning: a Laboratory Manual 4th edition, J. F. Sambrook and D. W. Russell, ed. Cold Spring Harbor Laboratory Press 2012; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the disclosure.

### General Description

One aspect of the disclosure is directed to a method for obtaining antibody-producing cells that express antibody molecules exhibiting high binding affinity for an antigen. A key feature of the present method involves an initial binding step which allows a first labeled form of the antigen to bind to antibody molecules on the surface of antibody-producing cells and form an antigen-antibody complex, followed by a “chase” step where the cells are contacted with one of several forms of the antigen: (i) an unlabeled form of the antigen (“cold chase”), (ii) a second labeled form of the antigen (“hot chase”), or an unlabeled form of the antigen and a second labeled form of the antigen (“combination chase”). The cells that remain bound to the first labeled form of the antigen after the chase represent cells expressing antibody molecules with high binding affinity.

Accordingly, the methods disclosed herein permit selection among cells expressing antibody molecules with different affinities to enrich for cells that express antibody molecules with high affinities.

The term “antibody molecule”, as used herein, encompasses both full length antibodies and antigen-binding fragments thereof (e.g., Fab, Fab′, and F(ab′)₂).

The term “enriching” means increasing the frequency or percentage of desired cells in a cell population, e.g., increasing the percentage of antibody-producing cells expressing high affinity antibody molecules within an antibody-producing cell population containing cells expressing antibody molecules having various affinities (e.g., high affinity, medium affinity, and low affinity). Thus, an antibody-producing cell population enriched in cells expressing high affinity antibody molecules encompasses an antibody-producing cell population having a higher frequency and/or higher percentage of antibody-producing cells expressing high affinity antibody molecules as a result of an enrichment process. In the present context, the enrichment process is a process that includes an antigen chase, thereby selecting cells that express high affinity antibody molecules to an antigen of interest from a population of cells that express antibody molecules of various affinities to the antigen of interest, and separating cells that express high affinity antibody molecules from cells that express antibody molecules not of high affinity.

The cell population obtained as a result of the disclosed method is enriched in antibody-producing cells expressing antibody molecules with high binding affinity to an antigen of interest. In other words, the enriched population of cells (cells collected after a chase) contains a greater percentage of cells that express an antibody molecule that binds to the antigen of interest with high binding affinity, as compared to a cell population before or without the chase. In some embodiments, at least 40% of the cells collected express a high affinity antibody molecule, e.g., antibody molecules having a KD from 0.1 pM to 25 nM. In some embodiments, the enriched cell population may be a population having at least 50% of cells within the population expressing an antibody molecule that binds to the antigen of interest with high binding affinity, e.g., antibody molecules having a KD from 0.1 pM to 25 nM. In some embodiments, the enriched cell population may be a population having at least 60% of cells within the population expressing an antibody molecule that binds to the antigen of interest with high binding affinity, e.g., antibody molecules having a KD from 0.1 pM to 25 nM. In some embodiments, the enriched cell population may be a population having at least 70% of cells within the population expressing an antibody molecule that binds to the antigen of interest with high binding affinity, e.g., antibody molecules having a KD from 0.1 pM to 25 nM. In some embodiments, the enriched cell population may be a population having at least 80% of cells within the population expressing an antibody molecule that binds to the antigen of interest with high binding affinity, e.g., antibody molecules having a KD from 0.1 pM to 25 nM. In some embodiments, the enriched cell population may be a population having at least 90% of cells within the population expressing an antibody molecule that binds to the antigen of interest with high binding affinity, e.g., antibody molecules having a KD from 0.1 pM to 25 nM. In some embodiments, the enriched cell population may be a population having at least 95% of cells within the population expressing an antibody molecule that binds to the antigen of interest with high binding affinity, e.g., antibody molecules having a KD from 0.1 pM to 25 nM. In some embodiments, the frequency of cells expressing high affinity antibody molecules (e.g., antibody molecules having a KD from 0.1 pM to 25 nM) in the cell population after a chase is increased by at least 30% as compared to the frequency in the cell population before or without the chase. In some embodiments, the frequency of cells expressing high affinity antibody molecules (e.g., antibody molecules having a KD from 0.1 pM to 25 nM) in the cell population after a chase is increased by at least 40% as compared to the frequency in the cell population before or without the chase. In some embodiments, the frequency of cells expressing high affinity antibody molecules (e.g., antibody molecules having a KD from 0.1 pM to 25 nM) in the cell population after a chase is increased by at least 50% as compared to the frequency in the cell population before or without the chase. In some embodiments, the frequency of cells expressing high affinity antibody molecules (e.g., antibody molecules having a KD from 0.1 pM to 25 nM) in the cell population after a chase is increased by at least 75% as compared to the frequency in the cell population before or without the chase. In some embodiments, the frequency of cells expressing high affinity antibody molecules (e.g., antibody molecules having a KD from 0.1 pM to 25 nM) in the cell population after a chase is increased by at least 100% as compared to the frequency in the cell population before or without the chase. In some embodiments, the frequency of cells expressing high affinity antibody molecules (e.g., antibody molecules having a KD of 0.1 pM to 25 nM) in the cell population after a chase is increased by at least 200% as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 40% as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 50% as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 75% as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 100% as compared to the frequency in the cell population before or without the chase. In some embodiments, the frequency of cells expressing high affinity antibody molecules having a KD of less than 1 nM (e.g., antibody molecules having a KD from 0.1 pM to 1 nM) in the cell population after a chase is increased by at least 200% as compared to the frequency in the cell population before or without the chase. In some embodiments, the frequency of cells expressing high affinity antibody molecules having a KD of less than 0.1 nM (e.g., antibody molecules having a KD from 0.1 pM to 0.1 nM) in the cell population after a chase is increased by at least 40% as compared to the frequency in the cell population before or without the chase. In some embodiments, the frequency of cells expressing high affinity antibody molecules having a KD of less than 0.1 nM (e.g., antibody molecules having a KD from 0.1 pM to 0.1 nM) in the cell population after a chase is increased by at least 50% as compared to the frequency in the cell population before or without the chase. In some embodiments, the frequency of cells expressing high affinity antibody molecules having a KD of less than 0.1 nM (e.g., antibody having a KD from 0.1 pM to 0.1 nM) in the cell population after a chase is increased by at least 75% as compared to the frequency in the cell population before or without the chase. In some embodiments, the frequency of cells expressing high affinity antibody molecules having a KD of less than 0.1 nM (e.g., antibody molecules having a KD from 0.1 pM to 0.1 nM) in the cell population after a chase is increased by at least 100% as compared to the frequency in the cell population before or without the chase. In some embodiments, the frequency of cells expressing high affinity antibody molecules having a KD of less than 0.1 nM (e.g., antibody having a KD from 0.1 pM to 0.1 nM) in the cell population after a chase is increased by at least 200% as compared to the frequency in the cell population before or without the chase.

In some embodiments, the method for obtaining antibody-producing cells that express antibody molecules exhibiting high binding affinity for an antigen comprises the following steps:
(a) contacting a population of antibody-producing cells that express antibody molecules to the antigen on the cell surface, with a first labeled form of the antigen to allow the antigen to bind to the antibody molecules on the cell surface, wherein the antigen of the first labeled form is conjugated to a first detectable label;
(b) washing the cells to remove unbound antigen;
(c) contacting the cells with either (i) an unlabeled form of the antigen, (ii) a second labeled form of the antigen, or (iii) the unlabeled form of the antigen and the second labeled form of the antigen;
(d) washing the cells to remove unbound antigen;
(e) collecting cells that remain bound to the first labeled form of the antigen, thereby obtaining a population of cells enriched in cells expressing high affinity antibody molecules.
   Antibody Molecules with High Binding Affinity

“Binding affinity,” as that term is known in the art, generally refers to the strength of the sum total of noncovalent interactions between a single binding site of a molecule (e.g., an antibody or fragment thereof) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule for its binding partner can generally be represented by the dissociation equilibrium constant (KD or K_{D}). There is an inverse relationship between K_{D} (molar) value and binding affinity, therefore the smaller the K_{D} value (M), the higher the affinity. Thus “higher affinity” refers to antibody molecules that generally bind antigen stronger and/or faster and/or remain bound longer. Generally, a lower concentration (M) of antigen is needed to achieve the desired effect due to its stronger binding interaction.

The term “kd” (sec-1 or 1/s) refers to the dissociation rate constant of a particular antibody-antigen interaction, or the dissociation rate constant of an antibody, Ig, antibody-binding fragment, or molecular interaction. This value is also referred to as the k_{off} value.

The term “ka” (M-1×sec-1 or 1/M) refers to the association rate constant of a particular antibody-antigen interaction, or the association rate constant of an antibody, Ig, antibody-binding fragment, or molecular interaction.

The term “KD” or “K_{D}” (M) refers to the equilibrium dissociation constant of a particular antibody-antigen interaction, or the equilibrium dissociation constant of an antibody, Ig, antibody-binding fragment, or molecular interaction. The equilibrium dissociation constant is obtained by dividing the ka with the kd.

A variety of methods of measuring binding affinity are known in the art, any of which can be used for purposes of the present invention. Binding affinities obtained using the method are typically in the range of about 0.1 pM to about 25 nM as determined by surface plasmon resonance. In some embodiments, binding affinities are less than about 10 nM as determined by surface plasmon resonance.

The term “high affinity” antibody molecule refers to those antibody molecules having a binding affinity, expressed as KD, of 25 nM or less, e.g., having a numerical value of about 0.1 pM to 25 nM. To this end, high affinity antibody molecules may have a measured KD about 25×10⁻⁹ M (25 nM) or less, about 10×10⁻⁹ M (10 nM) or less, about 1×10⁻⁹ M (1 nM) or less, about 1×10⁻¹⁰ M (0.1 nM) or less, about 0.5×10⁻¹⁰ M (0.05 nM) or less, about 0.05×10⁻¹⁰ M (5 pM) or less, about 1 pM or less, or about 0.5 pM or less, as measured by surface plasmon resonance, e.g., BIACORE™ or solution-affinity ELISA. Those of skill in the art will recognize that values for KD of antibody molecules may be represented numerically either as nE^{-z}, or as n×10^{-z}, for example, 3.2E⁻¹² is equivalent to 3.2×10⁻¹² and indicates a KD of 3.2 picomolar (pM). In some embodiments, the high affinity antibody molecules have a measured KD in a range of from about 0.1 pM to about 25 nM. In some embodiments, the high affinity antibody molecules have a measured KD in a range of from about 0.1 pM to about 20 nM. In some embodiments, the high affinity antibody molecules have a measured KD in a range of from about 0.1 pM to about 15 nM. In some embodiments, the high affinity antibody molecules have a measured KD in a range of from about 0.1 pM to about 10 nM. In some embodiments, the high affinity antibody molecules have a measured KD in a range of from about 0.1 pM to about 5 nM. In some embodiments, the high affinity antibody molecules have a measured KD in a range of from about 0.1 pM to about 1 nM. In some embodiments, the high affinity antibody molecules have a measured KD in a range of from about 0.1 pM to about 0.5 nM. In some embodiments, the high affinity antibody molecules have a measured KD in a range of from about 0.1 pM to about 0.1 nM. In some embodiments, the high affinity antibody molecules have a measured KD of less than about 20 nM. In some embodiments, the high affinity antibody molecules have a measured KD of less than about less than about 15 nM. In some embodiments, the high affinity antibody molecules have a measured KD of less than about 10 nM. In some embodiments, the high affinity antibody molecules have a measured KD of less than about 5 nM. In some embodiments, the high affinity antibody molecules have a measured KD of less than about 1 nM. In some embodiments, the high affinity antibody molecules have a measured KD of less than about 0.1 nM. In some embodiments, the high affinity antibody molecules have a measured KD of less than about 0.5 nM. In some embodiments, the high affinity antibody molecules have a measured KD of less than about 0.01 nM. In some embodiments, the high affinity antibody molecules have a measured KD of less than about 0.001 nM (or 1 pM). In some embodiments, the high affinity antibody molecules have a measured KD of less than about 0.5 pM.

### Antibody-Producing Cells

The terms “antibody-producing cells” and “antibody-expressing cells” refer to cells that express antibody molecules on the cell surface, i.e., the antibody molecules are bound to or anchored in the cell membrane. Cell surface expression of antibody molecules can occur either naturally, e.g., as a result of B-cell activation or as a result of recombinant technology and genetic engineering. The term, therefore, encompasses lymphocytes of antigen-dependent B-cell lineage, including memory B-cells, recombinant cells such as non-lymphoid cells engineered to express antibody molecules on the cell surface, including yeast and mammalian cells such as immortalized cells and hybridoma cells. In some embodiments, the immortalized cells include Chinese hamster ovary (CHO) cells, human embryonic kidney (HEK)293 cells, and murine myeloma cells (e.g., NS0 and Sp2/0).

In some embodiments, antibody-producing cells are primary antibody-producing cells. “Primary cells” refers to cells grown outside of their natural environment, such as tissue cells isolated from a mammal. In certain embodiments, primary antibody-producing cells are tissue-derived, such as from spleen, lymph node, bone marrow, or peripheral blood. In some embodiments, antibody-producing cells may be derived from primary antibody-producing cells. For example, primary antibody-producing cells may be fused to myeloma cells to make hybridomas, or otherwise immortalized, such as infected with a virus (e.g., EBV), or may be differentiated by cell sorting techniques based on protein markers expressed by particular B cell types.

In some embodiments, antibody-producing cells are mammalian cells or yeast engineered to express antibody molecules on the cell surface. In the context of cells engineered to express antibody molecules, the cells may be engineered to express full length immunoglobulin molecules or antigen-binding fragments. In some embodiments, antibody-producing cells are yeast cells (e.g., S. cerevisiae or Pichia) engineered to express antibody molecules on the cell surface. In some embodiments, antibody-producing cells are mammalian cells engineered to express antibody molecules on the cell surface. In some embodiments, the mammalian cells are immortalized cells engineered to express antibody molecules on the cell surface, which include, e.g., CHO cells, HEK293 cells, and murine myeloma cells (e.g., NS0 and Sp2/0).

Some methodologies described herein also apply to cell surface display platforms of various host cells, including yeast or mammalian cells such as CHO cells, that express antibody molecules on the cells surface to permit screening from antibody gene libraries or repertoires of antibody maturation variants.

Yeast surface display (YSD) platforms have been described and widely used in antibody screening (Border and Wittrup, Nat Biotechnol. 1997; 15:553-7; Feldhaus M J et al., Nat Biotechnol. 2003; 21:163-70; McMahon C et al., Nat Struct Mol Biol. 2018; 25:289-96). Display of Fab regions on the yeast surface has been reported to increase the antibody diversity and enlarge the library size (Weaver-Feldhaus J M et al., FEBS Lett. 2004; 564: 24-34; Rosowski S et al., Microb Cell Fact. 2018; 17:3; Sivelle C et al., MAbs. 2018; 10:720-9).

Mammalian cell surface display platforms that display full-length antibodies or Fab fragments on the surface of mammalian cells, including CHO cells, have been described, e.g., by Zhou et al. MAbs. 2010; 2(5): 508-518; Nguyen et alt, Protein Engineering, Design & Selection, 2018, vol. 31 no. 3. pp. 91-101). In addition, suitable for use herein are mammalian cells that carry a single antibody gene can be transfected with a gene encoding activation induced deaminase (AID) which initiates somatic hypermutation (SHM) by converting deoxycytidines (dC) to deoxyuracils (dU), to mutate the antibody gene in cells during cell proliferation in the cell culture. See, e.g., Chen C. et al., Biotechnol Bioeng. 113, 39-51 (2016).

### Immunization and Collection of Primary Antibody-Producing Cells

Immunization of mammals including human and nonhuman animals can be done by any methods known in the art (see, for example, E. Harlow and D. Lane—Antibodies A Laboratory Manual, Cold Spring Harbor (1988); Malik and Lillehoj, Antibody techniques: Academic Press, 1994, CA). The antigen of interest is administered as a protein, protein fragment, protein-fusion or DNA plasmid that contains the antigen gene of interest and expressing the antigen of interest using the host cellular expression machinery to express the antigen polypeptide in vivo. It is understood that the immunized mammal may be a human having been exposed to antigen and expressing humoral immunity for the antigen of interest. Antigen may be administered directly to a mammal, without adjuvant, or with adjuvant to aid in stimulation of the immune response. Adjuvants known in the art include, but are not limited to, complete and incomplete Freund's adjuvant, MPL+TDM adjuvant system (Sigma), or RIBI (muramyl dipeptides) (see O'Hagan, Vaccine Adjuvant, by Human Press, 2000, NJ). Without relying on a particular theory, adjuvant can prevent rapid dispersal of polypeptide by sequestering the antigen in a local depot and may contain factors that can stimulate host immune response.

Once an appropriate immune response has been achieved, antibody-producing cells are collected from the immunized animal.

Antibody-producing cells can be collected from different sources of an immunized animal, including but not limited to spleen, lymph node, bone marrow, and peripheral blood. In some embodiments, following immunization, splenocytes are harvested from an immunized animal. In some embodiments, peripheral blood mononuclear cells (PBMCs) are harvested from an immunized animal.

In some embodiments of the methods, a population of antibody-producing cells are antibody-producing B cells. In some embodiments, antibody-producing B cells can be obtained from immunized animals and isolated by FACS based on cell-surface B cell markers. B cell markers are known in the art. For example, applicable B cell markers that can be detected through the use of FACS include, but are not limited to, IgG, IgM, IgE, IgA, IgD, CD1, CD5, CD19, CD20, CD21, CD22, CD23, CD24, CD25, CD27, CD30, CD38, CD40, CD78, CD80, CD138, CD319, TLR4, IL-6, PDL-2, CXCR3, CXCR4, CXCR5, CXCR6, IL-10, and TGFβ.

In some embodiments, following immunization, splenocytes are harvested from an immunized animal. Following removal of red blood cells by lysis, IgG+antigen-positive B cells can be isolated and used as a antibody producing cell population in the method.

In some embodiments, peripheral blood mononuclear cells (PBMCs) are harvested from an immunized animal known to have humoral immunity to an antigen of interest. IgG+, antigen-positive B cells can then be isolated for use as primary antibody producing cells in the present method.

Collected primary antibody-producing cells, such as antibody-producing B cells, can be processed to enrich for cells that express antibody on the cell surface that is directed to an antigen of interest. In some embodiments, an initial step of purification is optionally performed to enrich for primary antibody-producing cells. Such purification includes affinity chromatography, also referred to as affinity purification. There are several types of affinity purification known in the art, such as ammonium sulfate precipitation, affinity purification with immobilized protein A, G, A/G, or L; and affinity purification with immobilized antigen.

### Antigens of Interest

The methods disclosed herein are available for use with any antigen of interest. An antigen of interest is any substance that results in an immune response. In some embodiments, the antigen of interest is a soluble protein. In some embodiments, the antigen of interest is a transmembrane protein. The antigen of interest can be any substance to which an antibody can bind including, but not limited to, peptides, proteins or fragments thereof; carbohydrates; organic and inorganic molecules; receptors produced by animal cells, bacterial cells, and viruses; enzymes; agonists and antagonists of biological pathways; hormones; and cytokines. Exemplary antigens include, but are not limited to, IL-2, IL-4, IL-6, IL-10, IL-12, IL-13, IL-18, IFN-α, IFN-γ, Angiotensin II, BAFF, CGRP, CXCL13, IP-10, PCSK9, NGF, Nav1.7, VEGF, EPO, EGF, and HRG. In some embodiments, the antigen of interest is a cytokine. In some embodiments, the antigen of interest is a growth factor. In some embodiments, the antigen of interest may be a tumor marker. In some embodiments, the antigen of interest is a viral protein. In some embodiments, the antigen of interest is a surface protein from a pathogen. In some embodiments, the antigen of interest is an interleukin (IL). In some embodiments, the antigen of interest is IL-13.

In some embodiments, the antigen is a protein that is present in a monomeric form. Examples of proteins that exist in monomers include interleukin molecules, such as IL-13. In some embodiments, the antigen is a protein that is present in a multimeric form, including homomers and heteromers. In some embodiments, the antigen is a protein that is present in both monomeric and multimeric forms, in which case a mixture of protein monomers and multimers can be used in the method described herein.

Whether the antigen is a protein that is present in a monomeric form, multimeric form, or a mixture there, the antigen may be employed in the methods described herein in a monovalent form or a multivalent form. The terms “monovalent” and “multivalent” are used to refer to the number of units of antigen being presented and to differentiate from the antigen itself being a protein in a monomer form, a multimer form or a mixture thereof. Thus, a monovalent form of an antigen refers to a single unit form of the antigen, where the antigen itself may be a protein in a monomeric form, a multimeric form or a mixture thereof. A multivalent form of an antigen refers to multiple units of the antigen being presented, typically by way of a multivalent molecule to which the antigen is bound or linked. A multivalent molecule can be a dimer, trimer, tetramer, pentamer, hexamer, and the like, or a combination thereof. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer includes tetramer and may additionally include trimer and/or dimer. In some embodiments, a streptavidin multimer is conjugated with a fluorophore such as phycoerythrin. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

### Initial Binding Step and Antigen Labels

To select for the cells that express antibody molecules exhibiting the highest binding affinity for an antigen of interest, an initial binding or contacting step is performed. In this step, the antibody-producing cells are contacted with a first labeled form of an antigen to allow the antigen to bind to antibody molecules on the cell surface.

In some embodiments, the first labeled form of the antigen is at a concentration between 0.001 nM and 1 μM. In some embodiments, the first labeled form of the antigen is at a concentration between 0.01 nM and 100 nM. In some embodiments, the first labeled form of the antigen is at a concentration between 0.05 nM to 10 nM. In some embodiments, the first labeled form of the antigen is at a concentration between 0.05 nM to 9 nM, 0.05 nM to 8 nM, 0.05 nM to 7 nM, 0.05 nM to 6 nM, 0.05 nM to 5 nM, 0.05 nM to 4 nM, 0.05 nM to 3 nM, 0.05 nM to 2 nM, or 0.05 nM to 1 nM. In some embodiments, the first labeled form of the antigen is at a concentration between 0.1 nM to 7.5 nM. In some embodiments, the first labeled form of the antigen is at a concentration between 0.1 nM to 7 nM, 0.1 nM to 6 nM, 0.1 nM to 5 nM, 0.1 nM to 4 nM, 0.1 nM to 3 nM, 0.1 nM to 2 nM, or 0.1 nM to 1 nM. In some embodiments, the first labeled form of the antigen is at a concentration between 0.2 nM to 7.5 nM. In some embodiments, the first labeled form of the antigen is at a concentration between 0.2 nM to 7 nM, 0.2 nM to 6 nM, 0.2 nM to 5 nM, 0.2 nM to 4 nM, 0.2 nM to 3 nM, 0.2 nM to 2 nM, 0.2 nM to 1 nM, 0.3 nM to 7 nM, 0.3 nM to 6 nM, 0.3 nM to 5 nM, 0.3 nM to 4 nM, 0.3 nM to 3 nM, 0.3 nM to 2 nM, 0.3 nM to 1 nM, 0.5 nM to 7 nM, 0.5 nM to 6 nM, 0.5 nM to 5 nM, 0.5 nM to 4 nM, 0.5 nM to 3 nM, 0.5 nM to 2 nM, 0.5 nM to 1 nM. In some embodiments, the first labeled form of the antigen is at a concentration between 1.0 nM to 10 nM, 1.0 nM to 9 nM, 1.0 nM to 8.0 nM, 1.0 nM to 7 nM, 1.0 nM to 6 nM, 1.0 nM to 5 nM, 1.0 nM to 4 nM, 1.0 nM to 3 nM, 1.0 nM to 2 nM, 2.0 nM to 10.0 nM, or 5.0 nM to 10.0 nM. In specific embodiments, the antibody-producing cells can be contacted with a first labeled form of the antigen where the first labeled form of the antigen is at a concentration of 0.2 nM. In specific embodiments, the antibody-producing cells can be contacted with a first labeled form of the antigen where the first labeled form of the antigen is at a concentration of 5.0 nM. In specific embodiments, the antibody-producing cells can be contacted with a first labeled form of the antigen where the first labeled form of the antigen is at a concentration of 7.5 nM. In specific embodiments, the antibody-producing cells can be contacted with a first labeled form of the antigen where the first labeled form of the antigen is at a concentration of 10 nM. In specific embodiments, the antibody-producing cells can be contacted with a first labeled form of the antigen where the first labeled form of the antigen is at a concentration of 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 8.0 nM, 8.5 nM, 9.0 nM, 9.5 nM, or greater.

In some embodiments, the contacting of the antibody producing cells with a first labeled form of the antigen occurs from about 5 to about 60 minutes. In some embodiments, the contacting of the antibody producing cells with a first labeled form of the antigen occurs for about 30 minutes. In some embodiments, the contacting of the antibody producing cells with a first labeled form of the antigen occurs for about 20 minutes. In some embodiments, the contacting of the antibody producing cells with a first labeled form of the antigen occurs for about 40 minutes. In some embodiments, the contacting of the antibody-producing cells with a first labeled form of the antigen occurs for about 10 minutes. In some embodiments, the contacting of the antibody-producing cells with a first labeled form of the antigen occurs for about 50 minutes.

In some embodiments, the first labeled form of antigen is a monovalent form of the antigen. In some embodiments, the first labeled form of antigen is a multivalent form of the antigen. In some embodiments, the first labeled form of antigen is a mixture of monovalent and multivalent forms of the antigen. Whether a monovalent form, a multivalent form or a mixture thereof, the antigen itself can be a protein that is a monomer, multimer, or a mixture of a monomer and multimer.

In some embodiments, the first labeled form of the antigen is the antigen conjugated to a first detectable label. The antigen can be labeled with small molecules, radioisotopes, enzymatic proteins and fluorescent dyes. In some embodiments, the detectable label is a small molecule. Detectable small molecule labels allow for easy labeling of proteins and can be used in a number of regularly deployed detection assays known in the art.

In some embodiments, the detectable label is an enzyme reporter. Enzyme labels are larger than biotin, however, they rarely disrupt antibody function. Commonly used enzyme labels are horseradish peroxidase (HRP), alkaline phosphatase (AP), glucose oxidase and β-galactosidase. To use enzyme-labeled antibodies, samples are incubated with an enzyme-specific substrate that is catalyzed by the enzyme to produce a colored product (chromogenic assays) or light (chemiluminescent assays). Each enzyme has a set of substrates and detection methods that can be employed. For example, HRP can be reacted with diaminobenzidine to produce a brown-colored product or with luminol to produce light. In contrast, AP can be reacted with para-Nitrophenylphosphate (pNPP) to produce a yellow-colored product detected by a spectrophotometer or with 5-bromo-4-chloro-3-indolyl phosphate (BCIP) and nitroblue tetrazolium (NBT) to produce a purple-colored precipitate.

In some embodiments, the detectable label is a fluorescent label. Fluorescent labels are directly conjugated to the antibody, no enzyme/substrate or binding interactions are required for detection. Therefore, the amount of fluorescent signal detected is directly proportional to the amount of target protein in the sample. Fluorescent tags can be covalently attached to antibodies through primary amines or thiol.

Removal of Unbound Antigen after Initial Binding

After incubation of the antibody-producing cells with a first labeled form of the antigen (the initial binding step), any unbound antigen can be removed from the antibody-producing cells.

In some embodiments, the unbound antigen is removed through washing. As is known in the art, washing is a technique where a wash buffer is used to remove unwanted components. For the present disclosure, unwanted components include unbound antigen. The unbound antigen can be washed from the bound antigen in a non-limiting example by adding a wash buffer to the mixture of bound and unbound antigen, centrifuging the mixture, and removing the supernatant which comprises wash buffer and unbound antigen.

Wash buffers are known in the art. Wash buffers and wash steps are used to remove unbound and excessive components. Wash buffers can be designed for specific techniques, such as ELISA, immunoblotting, or immunohistochemistry. Some wash buffers are compatible with many different immunoassays. In some embodiments, the wash buffer is a phosphate buffered saline (PBS) based wash buffer. In some embodiments, the wash buffer is a Tris buffered saline (TBS) wash buffer. In some embodiments, the wash buffer comprises a detergent. In some embodiments, the detergent is Tween-20.

The cells are washed with wash buffer for an allotted time in order to remove unbound antigen. This allotted amount of time will be an amount of time sufficient to remove unbound antigen. In some embodiments, this allotted time can be from about 10 minutes to about 60 minutes to remove unbound antigen; multiple washes that total from 10 to 60 minutes may be used, e.g., 3 washes of 10 minutes or one 30-minute wash; 2-4 washes of 5-15 minutes each, etc. In one embodiment, washing the cells for a period of time comprising one (1) wash about 5 minutes, or about 10 minutes, or about 15 minutes, or about 20 minutes, or about 25 minutes, or about 30 minutes, or about 35 minutes, or about 40 minutes, or about 45 minutes, or about 50 minutes, or about 55 minutes, or about 60 minutes total, may be used. In some embodiments, washing the cells for a period of time comprising two (2) washes about 5 minutes each, or about 10 minutes each, or about 15 minutes each, or about 20 minutes each, or about 25 minutes each, or about 30 minutes each per wash, may be used. Additional washing intervals are contemplated, essentially equivalent to those described herein.

After washing and aspirating the supernatant comprising the wash buffer and unbound antigen, the pellet comprising cells bound with antigen can be used in subsequent steps. In some embodiments, the pellet comprising the bound antigen can be resuspended in a buffer and used in subsequent steps. In some embodiments, the buffer used to resuspend the pellet can be the same wash buffer. In some embodiments, the buffer used to resuspend the pellet can be a different buffer than the wash buffer.

### The Chase Step(s)

After contacting the antibody producing cells with the first labeled form of antigen (initial binding/contacting step) and once the unbound first labeled form of antigen is removed, the antibody-producing cells are contacted again, or “chased,” with the antigen to selectively enrich for cells expressing antibody molecules with high affinities to the antigen. This chase can be performed using any of the several forms of the antigen: (i) an unlabeled form of the antigen (“cold” chase), (ii) a second labeled form of the antigen (“hot” chase); or (iii) an unlabeled form of the antigen and a second labeled form of the antigen (a combination of a cold chase antigen and a hot chase antigen, also referred to as a “combination chase”). The chase allows the chase antigen to bind to the antibody molecules initially bound by the first labeled form of the antigen, thereby chasing the first labeled form of the antigen off the antibody molecules, unless the antibody molecule has high affinity for the antigen and remains bound to the first labeled form of the antigen after the chase.

In some embodiments, the chase is performed using an unlabeled form of the antigen (cold chase). In some embodiments, the unlabeled form of the antigen is a monovalent form of the antigen—that is, while the antigen itself can be a protein in a monomeric form, a multimeric form, or a mixture of monomeric and multimeric forms, a monovalent form of the antigen is the antigen itself without further, secondary multimerization. In some embodiments, the unlabeled form of the antigen is a multivalent form of the antigen, where the antigen itself can be a protein in a monomeric form, a multimeric form, or a mixture of monomeric and multimeric forms. In some embodiments, the unlabeled form of the antigen is a mixture of a monovalent form and a multivalent form of the antigen. In embodiments where a multivalent form of the antigen is used, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer may include trimer and/or dimer in addition to tetramer. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

In some embodiments, the chase is performed using a second labeled form of the antigen (hot chase). In such embodiments, after the initial binding and washing steps, the antibody-producing cells bound with the first labeled form of the antigen are chased with a second labeled form of the antigen. The second labeled form of the antigen has a label that provides a different detectable signal than the label on the first labeled form of the antigen. Suitable choices of the label have been described herein, as long as the label on the second labeled form of the antigen is different from the label on the first labeled form of antigen. Such labels include small molecules, radioisotopes, enzymatic proteins and fluorescent dyes. In some embodiments, the first label is AlexaFluor647, and the second label is phycoerythrin.

In some embodiments, the second labeled form of the antigen is a monovalent form of the antigen. In some embodiments, the second labeled form of the antigen is a multivalent form of the antigen. In some embodiments, the second labeled form of the antigen comprises a mixture of a monovalent and a multivalent form of the antigen. In embodiments where a multivalent form of the antigen is used, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (tetravalent) form of the antigen. In some embodiments, a streptavidin multimer may include trimer and/or dimer in addition to tetramer. In some embodiments, a streptavidin multimer is conjugated with a fluorophore such as phycoerythrin. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

The first labeled form of the antigen and the second labeled form of the antigen can be the same or different valent form of the antigen but must have labels that emit different detectable signals from each other. In some embodiments, the first labeled form of the antigen is a monovalent form of the antigen and the second labeled form of antigen is also a monovalent form. In some embodiments, the first labeled form of the antigen is a monovalent form of the antigen whereas the second labeled form of the antigen is a multivalent form.

In some embodiments, the chase is performed with an unlabeled form of an antigen (cold) and a second labeled form of the antigen (hot), also referred herein as a combination chase. In such embodiments, after the initial binding and washing steps, the antibody-producing cells are chased with an unlabeled form of the antigen and a second labeled form of the antigen. The two forms of chase antigen: the unlabeled form of the antigen (“cold chase antigen”) and the second labeled form of the antigen (“hot chase antigen”) can be brought into contact with the cells at the same time or sequentially (e.g., with the cold chase antigen being added first, followed by the hot chase antigen, or vice versa). In some embodiments, the unlabeled form of the antigen is a monovalent form. In some embodiments, the unlabeled form of the antigen is a multivalent form. In some embodiments, the unlabeled form of the antigen is a mixture of monovalent form and multivalent form. In some embodiments, the second labeled form of the antigen is a monovalent form. In some embodiments, the second labeled form of the antigen is a multivalent form. In some embodiments, the second labeled form of the antigen comprises a mixture of a monovalent form and a multivalent form. In embodiments where a multivalent form of the antigen is used in a combination chase, such form can be provided by a multivalent molecule to which the antigen is bound or linked. In some embodiments, the multivalent molecule is a streptavidin multimer (e.g., tetramer), which can be complexed with biotin which is then linked to the antigen, thereby providing a multivalent (e.g., tetravalent) form of the antigen. In some embodiments, a streptavidin multimer may include trimer and/or dimer in addition to tetramer. In some embodiments, the multivalent molecule is a dimer of an immunoglobulin Fc fragment, to which the antigen can be linked to provide a bivalent form of the antigen. In some embodiments, the multivalent molecule is a trimer of a trimerization molecule, such as foldon, to which the antigen can be linked to provide a trivalent form of the antigen.

For any format of the chase, the chase antigen concentration used is in excess as compared to the concentration of the first labeled form of the antigen, irrespective of the form of antigen used for the chase (i.e., unlabeled form, a second labeled form, or an unlabeled form and a second labeled form). In embodiments where an unlabeled form of the antigen is used (in a cold chase or in a combination chase), the antigen in the unlabeled form is at least 2 fold, i.e., 2 fold to up to, e.g., 2500 fold, in molar ratio relative to the first labeled form of the antigen. In some embodiments, the antigen in the unlabeled form is 2-fold in molar ratio relative to the first labeled form of the antigen. In some embodiments, the antigen in the unlabeled form is 3-fold in molar ratio relative to the first labeled form of the antigen. In some embodiments, the antigen in the unlabeled form is 4-fold in molar ratio relative to the first labeled form of the antigen. In some embodiments, the antigen in the unlabeled form is 5-fold in molar ratio relative to the first labeled form of the antigen. In some embodiments, the antigen in the unlabeled form is 6-fold in molar ratio relative to the first labeled form of the antigen. In some embodiments, the antigen in the unlabeled form is 7-fold in molar ratio relative to the first labeled form of the antigen. In some embodiments, the antigen in the unlabeled form is 8-fold in molar ratio relative to the first labeled form of the antigen. In some embodiments, the antigen in the unlabeled form is 9-fold in molar ratio relative to the first labeled form of the antigen. In some embodiments, the antigen in the unlabeled form is 10-fold in molar ratio relative to the first labeled form of the antigen. In some embodiments, the antigen in the unlabeled form is 15-fold in molar ratio relative to the first labeled form of the antigen. In some embodiments, the antigen in the unlabeled form is 20-fold in molar ratio relative to the first labeled form of the antigen. In some embodiments, the antigen in the unlabeled form is 25-fold in molar ratio relative to the first labeled form of the antigen. In some embodiments, the antigen in the unlabeled form is 30-fold in molar ratio relative to the first labeled form of the antigen. In some embodiments, the antigen in the unlabeled form is 35-fold in molar ratio relative to the first labeled form of the antigen. In some embodiments, the antigen in the unlabeled form is 40-fold in molar ratio relative to the first labeled form of the antigen. In some embodiments, the antigen in the unlabeled form is 45-fold in molar ratio relative to the first labeled form of the antigen. In some embodiments, the antigen in the unlabeled form is 50-fold in molar ratio relative to the first labeled form of the antigen. In some embodiments, the antigen in the unlabeled form is 60-fold in molar ratio relative to the first labeled form of the antigen. In some embodiments, the antigen in the unlabeled form is 70-fold in molar ratio relative to the first labeled form of the antigen. In some embodiments, the antigen in the unlabeled form is 80-fold in molar ratio relative to the first labeled form of the antigen. In some embodiments, the antigen in the unlabeled form is 90-fold in molar ratio relative to the first labeled form of the antigen. In some embodiments, the antigen in the unlabeled form is 100-fold in molar ratio relative to the first labeled form of the antigen. In some embodiments, the antigen in the unlabeled form is 110-fold in molar ratio relative to the first labeled form of the antigen. In some embodiments, the antigen in the unlabeled form is 120-fold in molar ratio relative to the first labeled form of the antigen. In some embodiments, the antigen in the unlabeled form is 130-fold in molar ratio relative to the first labeled form of the antigen. In some embodiments, the antigen in the unlabeled form is 140-fold in molar ratio relative to the first labeled form of the antigen. In some embodiments, the antigen in the unlabeled form is 150-fold in molar ratio relative to the first labeled form of the antigen. In some embodiments, depending on the concentration of the first labeled form of the antigen, the unlabeled form of the antigen may be at a concentration between 0.4 nM to 1 μM, or 10 to 600 nM. In some embodiments, depending on the concentration of the first labeled form of the antigen, the unlabeled form of the antigen may be at a concentration between 10 nM to 600 nM, 10 nM to 500 nM, 10 nM to 400 nM, 10 nM to 300 nM, 10 nM to 200 nM, 10 nM to 150 nM, 10 nM to 100 nM, 10 nM to 75 nM, 10 nM to 65 nM, 10 nM to 50 nM, 10 nM to 40 nM, 10 nM to 30 nM, 10 nM to 25 nM, or 10 nM to 20 nM. In embodiments where a second labeled form of the antigen is used (in a hot chase or a combination chase), the antigen in the second labeled form is at least 2 fold, i.e., 2 fold to up to, e.g., 2500 fold, in molar ratio relative to the first labeled form of the antigen. In some embodiments, the antigen in the second labeled form is 2-fold in molar ratio relative to the first labeled form of the antigen. In some embodiments, the antigen in the second labeled form is 3-fold in molar ratio relative to the first labeled form of the antigen. In some embodiments, the antigen in the second labeled form is 4-fold in molar ratio relative to the first labeled form of the antigen. In some embodiments, the antigen in the second labeled form is 5-fold in molar ratio relative to the first labeled form of the antigen. In some embodiments, the antigen in the second labeled form is 6-fold in molar ratio relative to the first labeled form of the antigen. In some embodiments, the antigen in the second labeled form is 7-fold in molar ratio relative to the first labeled form of the antigen. In some embodiments, the antigen in the second labeled form is 8-fold in molar ratio relative to the first labeled form of the antigen. In some embodiments, the antigen in the second labeled form is 9-fold in molar ratio relative to the first labeled form of the antigen. In some embodiments, the antigen in the second labeled form is 10-fold in molar ratio relative to the first labeled form of the antigen. In some embodiments, the antigen in the second labeled form is 15-fold in molar ratio relative to the first labeled form of the antigen. In some embodiments, the antigen in the second labeled form is 20-fold in molar ratio relative to the first labeled form of the antigen. In some embodiments, the antigen in the second labeled form is 25-fold in molar ratio relative to the first labeled form of the antigen. In some embodiments, the antigen in the second labeled form is 30-fold in molar ratio relative to the first labeled form of the antigen. In some embodiments, the antigen in the second labeled form is 35-fold in molar ratio relative to the first labeled form of the antigen. In some embodiments, the antigen in the second labeled form is 40-fold in molar ratio relative to the first labeled form of the antigen. In some embodiments, the antigen in the second labeled form is 45-fold in molar ratio relative to the first labeled form of the antigen. In some embodiments, the antigen in the second labeled form is 50-fold in molar ratio relative to the first labeled form of the antigen. In some embodiments, the antigen in the second labeled form is 60-fold in molar ratio relative to the first labeled form of the antigen. In some embodiments, the antigen in the second labeled form is 70-fold in molar ratio relative to the first labeled form of the antigen. In some embodiments, the antigen in the second labeled form is 80-fold in molar ratio relative to the first labeled form of the antigen. In some embodiments, the antigen in the second labeled form is 90-fold in molar ratio relative to the first labeled form of the antigen. In some embodiments, the antigen in the second labeled form is 100-fold in molar ratio relative to the first labeled form of the antigen. In some embodiments, the antigen in the second labeled form is 110-fold in molar ratio relative to the first labeled form of the antigen. In some embodiments, the antigen in the second labeled form is 120-fold in molar ratio relative to the first labeled form of the antigen. In some embodiments, the antigen in the second labeled form is 130-fold in molar ratio relative to the first labeled form of the antigen. In some embodiments, the antigen in the second labeled form is 140-fold in molar ratio relative to the first labeled form of the antigen. In some embodiments, the antigen in the second labeled form is 150-fold in molar ratio relative to the first labeled form of the antigen. In some embodiments, depending on the concentration of the first labeled form of the antigen, the second labeled form of the antigen may be at a concentration between 0.4 nM to 1 mM or 10 nM to 600 nM. In some embodiments, depending on the concentration of the first labeled form of the antigen, the second labeled form of the antigen may be at a concentration between 10 nM to 600 nM, 10 nM to 500 nM, 10 nM to 400 nM, 10 nM to 300 nM, 10 nM to 200 nM, 10 nM to 150 nM, 10 nM to 100 nM, 10 nM to 75 nM, 10 nM to 65 nM, 10 nM to 50 nM, 10 nM to 40 nM, 10 nM to 30 nM, 10 nM to 25 nM, or 10 nM to 20 nM.

In some embodiments where a combination chase is performed, the cold chase antigen and hot chase antigen can be at the same concentration or at different concentrations.

In some embodiments of a combination chase, the cells are contacted with a cold chase antigen and a hot chase antigen sequentially; and in some such embodiments, a washing step can be included between the incubations with the two chase antigens. In such embodiments, the cells are washed with wash buffer for an allotted time sufficient to remove unbound antigen. In some embodiments, washing the cells for a period of time comprising one (1) wash of about 10 minutes, or about 15 minutes, or about 20 minutes, or about 25 minutes, or about 30 minutes, or about 35 minutes, or about 40 minutes, or about 45 minutes, or about 50 minutes, or about 55 minutes, or about 60 minutes total, may be used. In some embodiments, washing the cells for a period of time comprising two (2) washes of about 5 minutes each, or about 10 minutes each, or about 15 minutes each, or about 20 minutes each, or about 25 minutes each, or about 30 minutes each per wash, may be used. Additional washing intervals are contemplated, essentially equivalent to those described herein.

The chase is performed for a period of time sufficient to allow the chase antigen to bind to antibodies. In some embodiments, the chase is performed with an unlabeled form of the antigen for a time of about 5 to about 60 minutes. In some embodiments, the chase is performed with an unlabeled form of the antigen for about 50 minutes. In some embodiments, the chase is performed with an unlabeled form of the antigen for about 45 minutes. In some embodiments, the chase is performed with an unlabeled form of the antigen for about 40 minutes. In some embodiments, the chase is performed for a time of about 30 minutes. In some embodiments, the chase is performed with an unlabeled form of the antigen for about 20 minutes. In some embodiments, the chase is performed with an unlabeled form of the antigen for about 10 minutes.

In some embodiments, the chase is performed with a second labeled form of the antigen for a time of about 5 to about 60 minutes. In some embodiments, the chase is performed with a second labeled form of the antigen for about 50 minutes. In some embodiments, the chase is performed with a second labeled form of the antigen for about 45 minutes. In some embodiments, the chase is performed with a second labeled form of the antigen for about 40 minutes. In some embodiments, the chase is performed with a second labeled form of the antigen for a time of about 30 minutes. In some embodiments, the chase is performed with a second labeled form of the antigen for about 20 minutes. In some embodiments, the chase is performed with a second labeled form of the antigen for about 10 minutes.

In some embodiments of a combination chase, the chase is performed with an unlabeled form of the antigen for a time of about 5 to about 60 minutes (e.g., 5 minutes, 10 minutes, 20 minutes, 30 minutes, 40 minutes, 45 minutes, 50 minutes, or 60 minutes), followed by contact with a second labeled form of the antigen for a time of about 5 to about 60 minutes (e.g., 5 minutes, 10 minutes, 20 minutes, 30 minutes, 40 minutes, 45 minutes, 50 minutes, or 60 minutes). In some embodiments of a combination chase, the chase is performed with a second labeled form of the antigen for a time of about 5 to about 60 minutes (e.g., 5 minutes, 10 minutes, 20 minutes, 30 minutes, 40 minutes, 45 minutes, 50 minutes, or 60 minutes), followed by an unlabeled form of the antigen for a time of about 5 to about 60 minutes (e.g., 5 minutes, 10 minutes, 20 minutes, 30 minutes, 40 minutes, 45 minutes, 50 minutes, or 60 minutes). The time of contact for the unlabeled form of antigen and the second labeled form of antigen can be the same or different length. As a non-limiting example, the unlabeled form of antigen may contact the primary antibody producing cells bound to a first labeled form of the antigen for about 30 minutes while the second labeled form of the antigen may subsequently contact the cells for about 45 minutes, and vice versa. In some embodiments of a combination chase, the chase is performed with an unlabeled form of the antigen and a second labeled form of the antigen at the same time for a time of about 5 to about 60 minutes, e.g., 5 minutes, 10 minutes, 20 minutes, 30 minutes, 40 minutes, 45 minutes, 50 minutes, or 60 minutes.

In some embodiments, the chase is performed more than once. In some embodiments, the cold chase is performed more than once. In some embodiments, the hot chase is performed more than once. In some embodiments, the cold chase is performed once followed by a hot chase performed more than once. In some embodiments, a cold chase is performed more than once followed by a hot chase performed once. In some embodiments, a cold chase is performed more than once followed by a hot chase performed more than once. In some embodiments, a combination chase is performed more than once. In some embodiments, a wash step is included after each chase step. In some embodiments, more than one wash step is included after each chase step, e.g., 2 washes or 3 washes.

Removal of Unbound Antigen after Chase

After a chase step (e.g., wherein the antibody-producing cells bound with a first labeled form of the antigen are chased with an unlabeled form of antigen, with the second labeled form of antigen, or with an unlabeled form of the antigen and a second labeled form of the antigen), unbound antigen is again removed.

In some embodiments, the unbound antigen is removed through washing.

In some embodiments, the chase and wash are repeated more than once before collecting the cells that remain bound to the first labeled form of the antigen, thereby obtaining a population of cells enriched in cells expressing high affinity antibodies.

### Fluorescence-Activated Cell Sorting (FACS)

Flow cytometry is a popular analytical cell-biology technique that utilizes light to count and profile cells in a heterogenous fluid mixture. Flow cytometry is a particularly powerful method because it allows a researcher to rapidly, accurately, and simply collect data related to many parameters from a heterogeneous fluid mixture containing live cells. Fluorescence-Activated Cell Sorting (FACS) is a derivative of flow cytometry that adds an exceptional degree of functionality. Using FACS a researcher can physically sort a heterogeneous mixture of cells into different populations.

Two-dimensional (2D) FACS is the sorting of cells based on two different fluorescent labels. Two-dimensional FACS provides more selective results than FACS based on one parameter. Two-dimensional FACS is available to be used in embodiments of the disclosure which use a hot chase. In some embodiments where the first detectable label is a first fluorescent label, and the second detectable label is a second fluorescent label that differentiates from the first fluorescent label, two-dimensional FACS is used to collect cells that remain bound to the first labeled form of the antigen. In specific embodiments, the first detectable label is A647 and the second detectable label is Phycoerythrin.

### Obtaining a Population of Cells Enriched in Cells Expressing High Affinity Antibody Molecules

The antibody-producing cells that remain bound to the first labeled form of the antigen after the chase are collected. This collecting of antibody-producing cells allows for obtaining a population of cells enriched in cells expressing high affinity antibody molecules.

The obtained population of cells enriched in cells expressing high affinity antibody molecules can be sorted or separated into single cells. In some embodiments, fluorescence-activated cell sorting (FACS) is used to sort and select single antibody-producing cells.

Protocols for single cell isolation by flow cytometry are well-known (Huang, J. et al, 2013, supra). Single antibody-producing cells may be sorted and collected by alternative methods known in the art, including but not limited to manual single cell picking, limited dilution, B cell panning of adsorbed antigen, microfluidics, laser capture microdissection, and Gel Bead Emulsions (GEMs), which are all well-known in the art. See, for example, Rolink et al., J Exp Med (1996)183:187-194; Lightwood, D. et al, J. Immunol. Methods (2006) 316(1-2):133-43; Gross et al., Int. J. Mol. Sci. (2015) 16: 16897-16919; and Zheng et al., Nature Communications (2017) 8: 14049. Gel Bead Emulsions (GEMs) are also commercially available (e.g., 10× Chromium System from 10× Genomics, Pleasanton, CA).

Once collected, single antibody-producing cells may be propagated by common cell culture techniques for subsequent DNA preparation. Alternatively, antibody genes may be amplified from single antibody-producing cells directly and subsequently cloned into DNA vectors.

Generating Antibodies from Nucleic Acids Obtained from Antibody-Producing Cells that Express High Affinity Antibody Molecules.

Nucleic acids encoding an antibody or a fragment thereof can be isolated from the antibody-producing cells obtained using the methods described herein.

In some embodiments, genes or nucleic acids encoding immunoglobulin variable heavy and variable light chains (i.e., VH and VL, and VL can be Vκ or Vλ chain) can be recovered using RT-PCR protocols with nucleic acids isolated from antibody-producing cells. These RT-PCR protocols are well known and conventional techniques, as described for example, by Wang et al., J. Immunol. Methods (2000) 244:217-225 and described herein.

In some embodiments, the nucleic acid encodes a fragment of an antibody, such as a variable domain, constant domain or combination thereof. In certain embodiments, the nucleic acid isolated from an antibody-producing cell encodes a variable domain of an antibody. In some embodiments, the nucleic acid encodes an antibody heavy chain or a fragment thereof (e.g., the variable domain of the antibody heavy chain). In other embodiments, the nucleic acid encodes an antibody light chain or a fragment thereof (e.g., the variable domain of the antibody light chain).

Once recovered, antibody-encoding genes or nucleic acids can be cloned into IgG heavy- and light-chain expression vectors and expressed via transfection of host cells. For example, antibody-encoding genes or nucleic acids can be inserted into a replicable vector for further cloning (amplification of the DNA) or for expression (stably or transiently) in cells. Many vectors, particularly expression vectors, are available or can be engineered to comprise appropriate regulatory elements required to modulate expression of an antibody encoding gene or nucleic acid.

An expression vector in the context of the present disclosure can be any suitable vector, including chromosomal, non-chromosomal, and synthetic nucleic acid vectors (a nucleic acid sequence comprising a suitable set of expression control elements) as described herein. Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In some embodiments, a nucleic acid molecule is included in a naked DNA or RNA vector, including, for example, a linear expression element (as described in, for instance, Sykes and Johnston, Nat Biotech (1997) 12:355-59), a compacted nucleic acid vector (as described in for instance U.S. Pat. No. 6,077,835), or a plasmid vector such as pBR322 or pUC 19/18. Such nucleic acid vectors and the usage thereof are well known in the art. See, for example, U.S. Pat. Nos. 5,589,466 and 5,973,972. In certain embodiments, the expression vector can be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH. See, F. Ausubel et al., ed. Current Protocols in Molecular Biology, Greene Publishing and Wiley InterScience New York (1987); and Grant et al., Methods in Enzymol 153, 516-544 (1987).

In certain embodiments, the vector comprises a nucleic acid molecule (or gene) encoding a heavy chain of the antibody and a nucleic acid molecule (or gene) encoding a light chain of the antibody, wherein the antibody is produced by an antibody-producing cell that has been obtained by a method of the present disclosure.

Host cells suitable for expression of antibody molecules include, but are not limited to, cells of either prokaryotic or eukaryotic (generally mammalian) origin. In some embodiments, the host cell is a bacterial or yeast cell. In some embodiments, the host cell is a mammalian cell. In other embodiments, the host cell can be, for example, a Chinese hamster ovarian cells (CHO) such as, CHO K1, DXB-11 CHO, Veggie-CHO cells; a COS (e.g., COS-7); a stem cell; retinal cells; a Vero cell; a CV1cell; a kidney cell such as, for example, a HEK293, a 293 EBNA, an MSR 293, an MDCK, aHaK, a BHK21 cell; a HeLa cell; a HepG2 cell; WI38; MRC 5; Colo25; HB 8065; HL-60; a Jurkat or Daudi cell; an A431 (epidermal) cell; a CV-1, U937, 3T3 or L-cell; a C127 cell, SP2/0, NS-0 or MMT cell, a tumor cell, and a cell line derived from any of the aforementioned cells. In a particular embodiment, the host cell is a CHO cell. In a specific embodiment, the host cell is a CHO K1 cell.

It will be appreciated that the full-length antibody nucleic acid sequence or gene may be subsequently cloned into an appropriate vector or vectors. Alternatively, the Fab region of an isolated antibody may be cloned into a vector or vectors in line with constant regions of any isotype. Therefore, any constant region may be utilized in the construction of isolated antibodies, including IgG1, IgG2, IgG3, IgG4, IgM, IgA, IgD, and IgE heavy chain constant regions, or chimeric heavy chain constant regions. Such constant regions can be obtained from any human or animal species depending on the intended use of the antibodies. Also, antibody variable regions or Fab region may be cloned in an appropriate vector(s) for the expression of the protein in other formats, such as ScFv, diabody, etc.

In some embodiments, host cells comprising one or more of antibody-encoding nucleic acids are cultured under conditions that express a full-length antibody, and the antibody can then be produced and isolated for further use. In certain embodiments, the host cell comprises a nucleic acid that encodes a variable domain of an antibody, and the cell is cultured under conditions that express the variable domain. In other embodiments, the host cell comprises a nucleic acid that encodes a variable heavy chain (VH) domain of an antibody, and the cell is cultured under conditions that express the VH domain. In another embodiment, the host cell comprises a nucleic acid that encodes a variable light chain (VL) domain of an antibody, and the cell is cultured under conditions that express the VL domain. In specific embodiments, the host cell comprises a nucleic acid that encodes a VH domain of an antibody and nucleic acid that encodes a VL domain of an antibody, and the cell is cultured under conditions that express the VH domain and the VL domain.

### EXAMPLES

### Example 1. Preparation of Beads

As a proof of concept, polystyrene microbeads were used in place of B cells. The polystyrene microbeads used were the same size as B cells. Polystyrene micro beads conjugated with polyclonal anti-hFc capture antibodies (Spherotech, Lake Forest, IL) were coated with four monoclonal antibodies against human IL13 protein with varying affinities: anti IL13-1, anti IL13-2, anti IL13-3, and anti IL13-4. The dissociation t_{1/2} of the binding between these four antibodies and hIL13 are 1.3 min for anti IL13-1, 10 min for anti IL13-2, 102 min for anti IL13-3, and 1155 min for anti IL13-4, as shown in Table 1.

TABLE 1

| Anti-hIL13 Abs | Ka (1/Ms) | Kd (1/s) | KD (M) | t½ (min) |
|---|---|---|---|---|
| anti IL13-1 | 1.42E+06 | 8.89E−03 | 6.26E−09 | 1.3 |
| anti IL13-2 | 1.25E+06 | 1.19E−03 | 9.53E−10 | 10 |
| anti IL13-3 | 6.27E+05 | 1.13E−04 | 1.81E−10 | 102 |
| anti IL13-4 | 2.03E+06 | 1.00E−05* | 4.93E−12 | 1155 |

The polystyrene beads were incubated with the four anti-human IL13 antibodies (anti IL13-1, anti IL13-2, anti IL13-3, and anti IL13-4) overnight at 4° C. The following day, the beads were washed with PBS to remove unbound antibodies. The conjugated beads were then incubated with A647 conjugated hIL13 at either 5 nM or 0.2 nM for 30 minutes. Staining profiles using 5 nM were preferable; however, concentrations throughout the range of 0.2 nM to 5 nM were also tested and successful. Unbound A647 conjugated hIL13 was removed through two washes using stain buffer, and the mAb coated beads were ready for use in chase experiments: no chase, monovalent cold chase, fluorophore labeled multivalent chase, and the combination chase with monomeric cold antigen and fluorophore labeled multivalent antigen.

### Example 2: Proof of Concept Separation of Polystyrene Beads in FACS Experiments Based on Antigen Binding Dissociation Rates

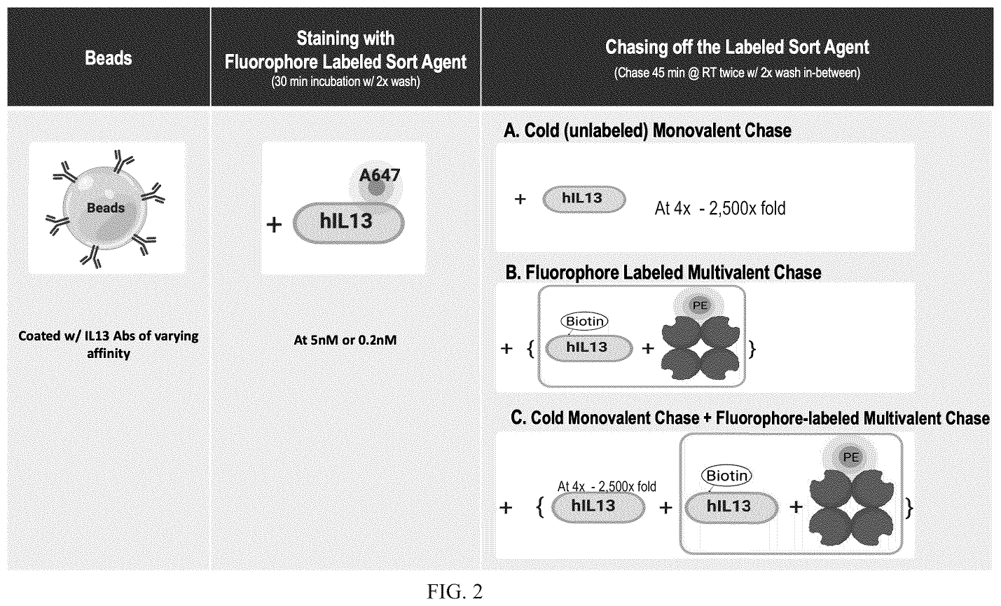

To demonstrate the ability to separate B cells in FACS-based experiments based on antigen binding dissociation rates, a fluorophore labeled antigen was used followed by variable chase steps: 1) no chase, 2) monovalent cold chase, 3) fluorophore labeled (“hot”) multivalent chase, and 4) combination chase with monovalent cold antigen and hot multivalent antigen. A schematic representation showing the overall view of the three exemplary chase methods used is shown in FIG. 2.

### No Chase

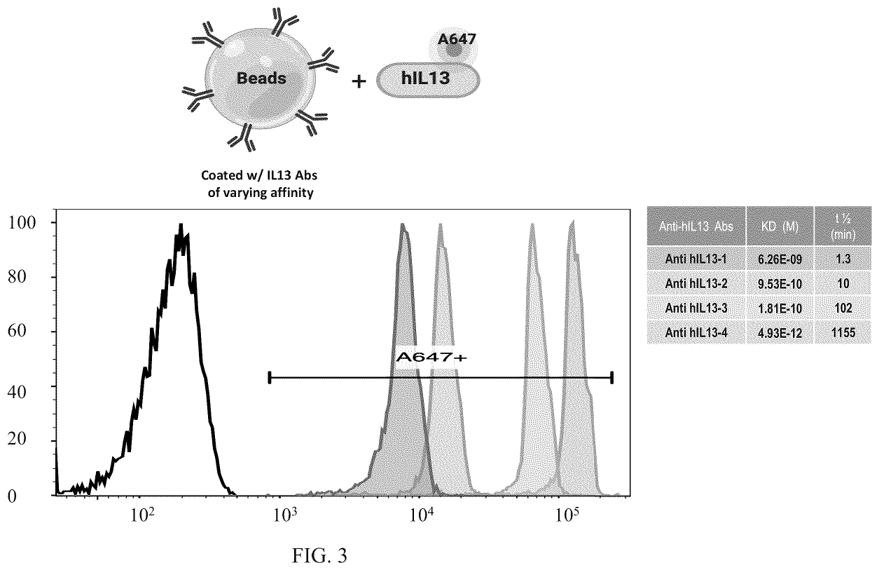

In the samples without chase, no further steps were needed but cells were pelleted by centrifugation, resuspended in PBS, and analyzed by flow cytometry. Results can be seen in FIG. 3, a representative univariate histogram overlay of the A647 staining of the IL13 antibody coated beads without chase, detected by flow cytometry.

### Monovalent Cold Chase

For monovalent cold chase experiments, the hIL13 mAb coated beads were incubated with 20 nM or 500 nM unlabeled hIL13 for 45 minutes, followed by two washes with PBS. A second incubation period with unlabeled hIL13 for another 45 minutes was performed. After the second incubation, the beads were pelleted by centrifugation, resuspended in PBS, and analyzed by flow cytometry.

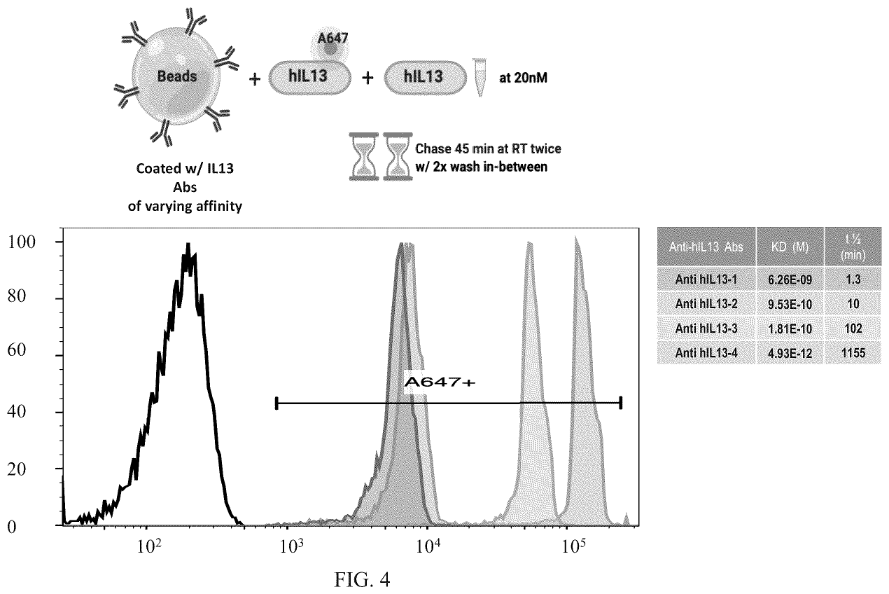

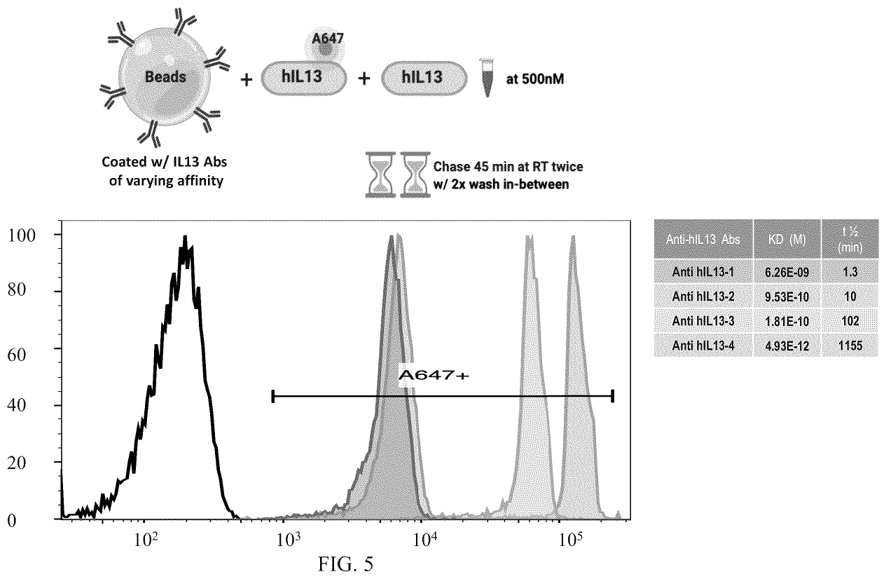

The results are shown in FIG. 4 and FIG. 5, which are representative univariate histogram overlay of A647 staining of the IL13 coated beads as detected by flow cytometry. FIG. 4 presents results for a monovalent cold chase experiment where four different groups of beads were coated with IL13 antibodies having different dissociation constants and were exposed to 5 nM A647 conjugated human IL13, washed, and were further incubated twice with 4× (20 nM) of unlabeled human IL13 for 45 minutes with two washes in-between, before the detection of A647 by flow cytometry. FIG. 5 presents results for a monovalent chase experiment where four different IL13 antibody coated beads were exposed to 5 nM A647 conjugated human IL13, washed, and were further incubated twice with 100× (500 nM) of unlabeled human IL13 for 45 minutes with two washes in-between. Beads coated with the two IL13 mAbs that have longer dissociation t_{1/2}s (anti IL13-3 and anti IL13-4) appeared at the same staining level with and without chase; while beads coated with the other two mAbs that have shorter t_{1/2}s (anti IL13-1 and anti IL13-2) show weaker staining and better separation from the beads coated with longer t_{1/2}s mAbs under cold chase condition than the samples without cold chase.

### Fluorophore Labeled Multivalent Chase

For the fluorophore labeled multivalent chase experiments, the hIL13 mAb coated beads were incubated with 20 nM biotin-hIL13 pre-bound with 5 nM Phycoerythrin (PE)-streptavidin (SA) for 45 minutes, followed by two washes of PBS before the second incubation of 20 nM biotin-hIL13 pre-bound with 5 nM PE-SA for another 45 minutes. After the second incubation, the beads were pelleted by centrifugation, resuspended in PBS, and analyzed by flow cytometry.

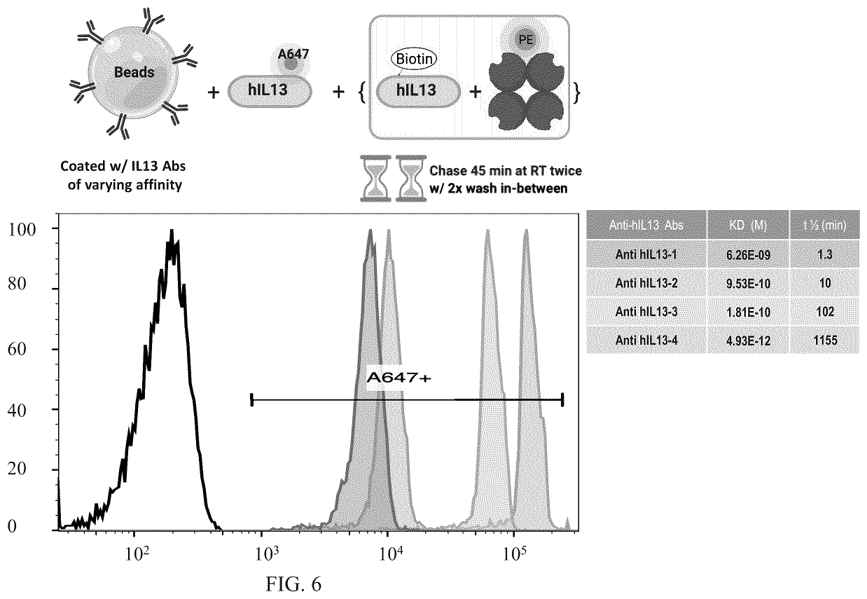

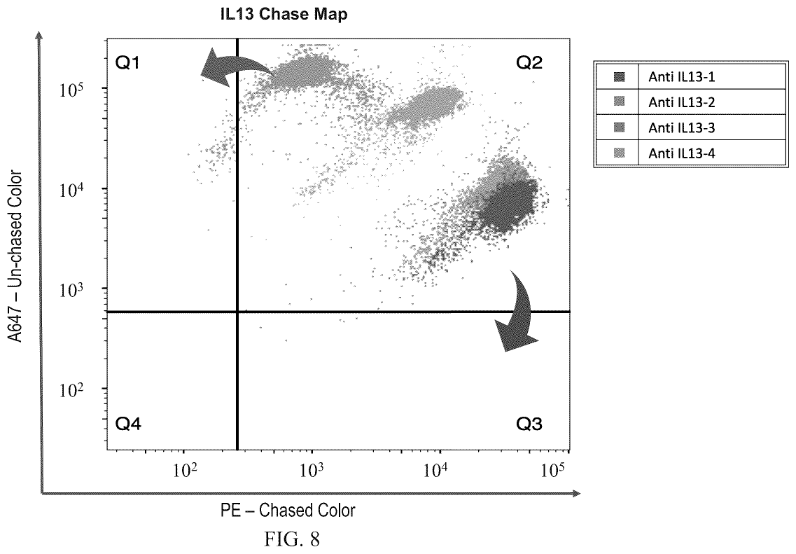

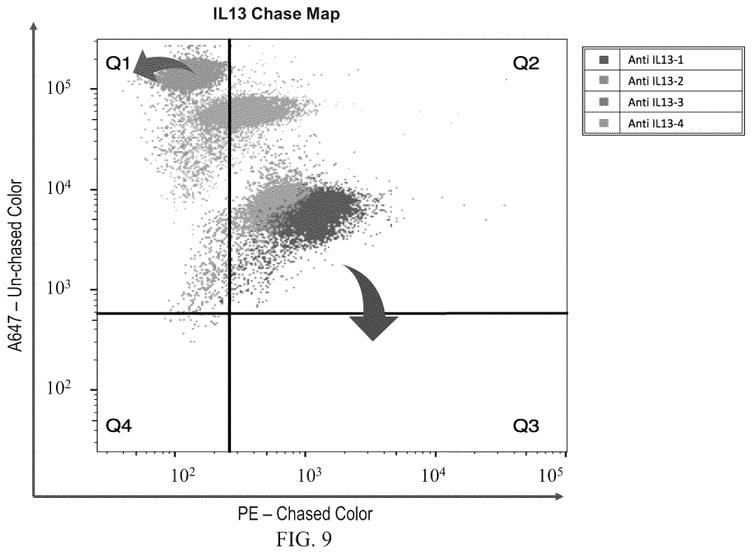

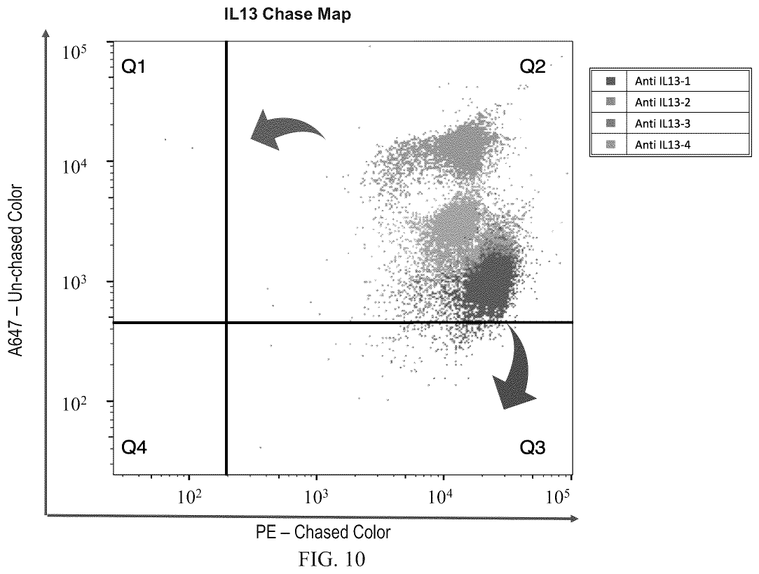

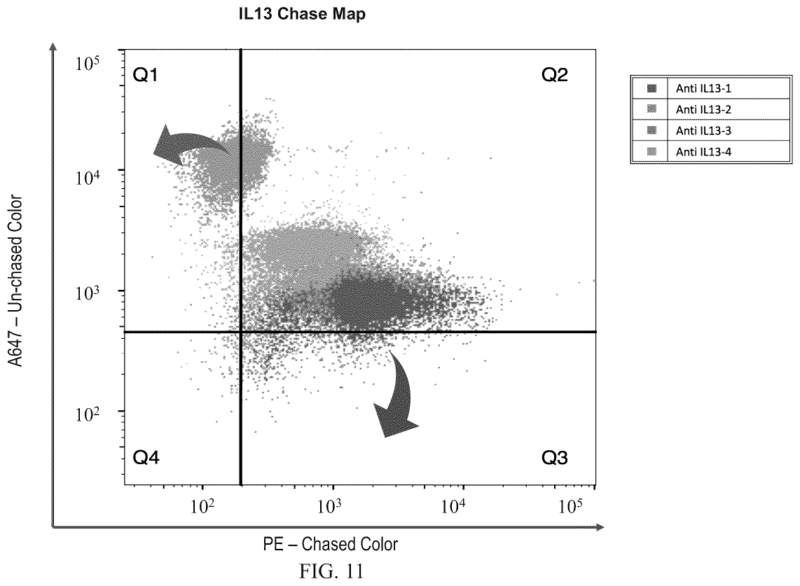

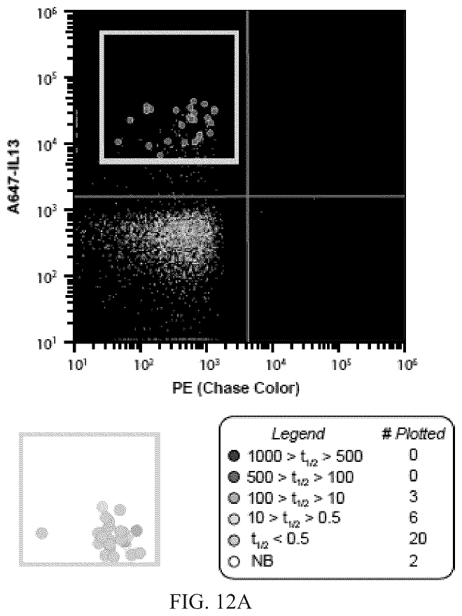

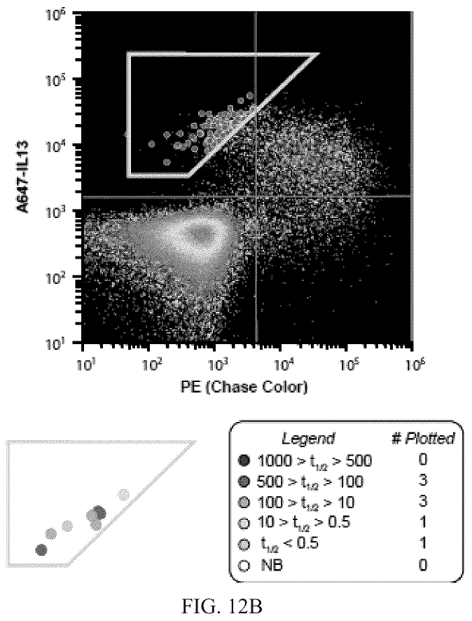

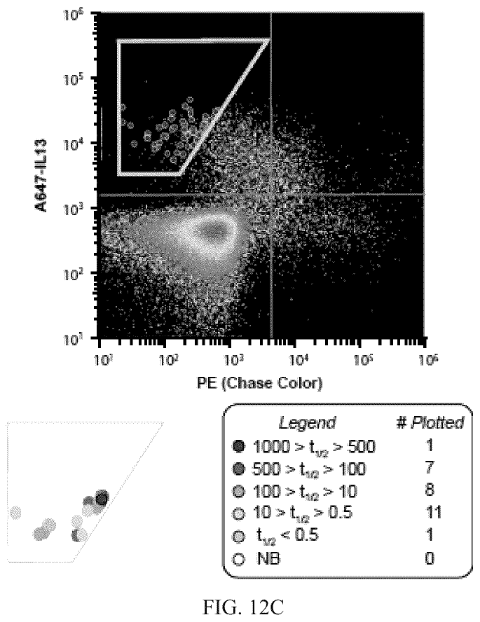

Fluorophore labeled multivalent chase experiment results are shown in both A647 histogram overlays (FIG. 6) as well as A647 vs PE 2-color dot plot overlays (FIG. 8 and FIG. 10). With A647 labeled hIL13 applied at 5 nM (as shown in FIG. 6), when looking only at A647 staining level, all mAb-coated beads that underwent fluorophore labeled multivalent chase showed the same differential A647-staining levels as their corresponding mAb-coated beads that underwent monomeric cold chase shown above; however, when looking at PE staining level, beads coated with mAb with the longest t_{1/2}s (t_{1/2}s=1155 min, anti IL13-4) showed the least PE staining, that further distances from the PE staining of beads coated with anti IL13-3 (t_{1/2}s=102 min), which is clearly separated from beads coated with anti IL13-1 and anti IL13-2, the two mAbs with shorter t_{1/2}s, 1.3 min and 10 min respectively, as shown in FIG. 8 and FIG. 10. The addition of the second color in this chase helped to further separate beads coated with the two longer t_{1/2}s from each other.

### Monovalent Cold Chase and Fluorophore Labeled Multivalent Chase Combination

For the monovalent cold chase and fluorophore labeled multivalent chase combination experiments, the hIL13 mAb coated beads were incubated with the combination of the above two chase agents, i.e., with 500 nM unlabeled hIL13 and 20 nM biotin-hIL13 pre-bound with 5 nM PE-streptavidin for 45 minutes, followed by two washes of PBS before a second incubation of 500 nM unlabeled hIL13 and 20 nM biotin-hIL13 pre-bound with 5 nM PE-streptavidin for another 45 minutes.

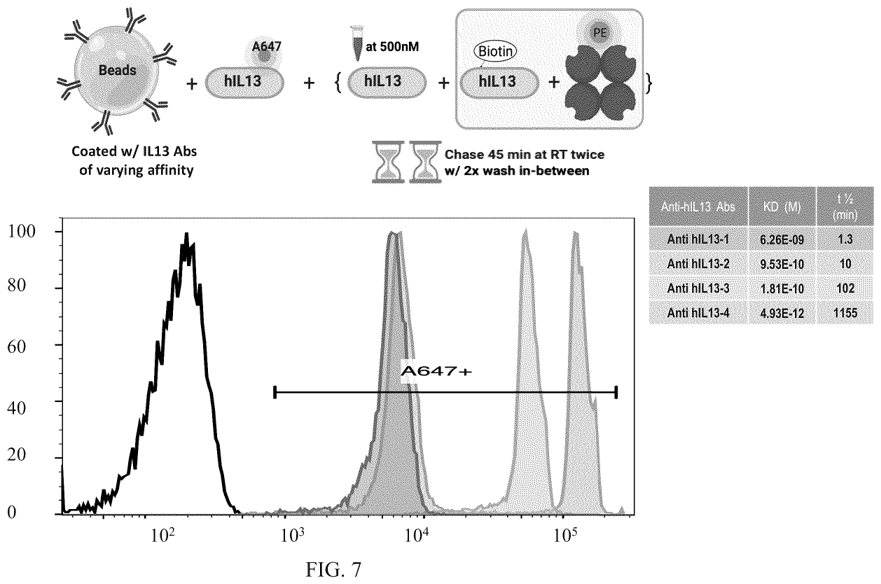

For the combination of monovalent cold chase and fluorophore labeled multivalent chase experiment, the results are again shown in both A647 histogram overlays (FIG. 7) as well as shown in A647 v PE 2-color dot plot overlays (FIG. 8 and FIG. 10). With A647 labeled hIL13 applied at 0.2 nM, beads coated with mAb that has the longest t_{1/2}s (t_{1/2}s=1155 min) clearly stands out and separated from the rest of the mAbs-coated beads in the A647 v PE dot plot overlay. Beads coated with anti IL13-3 (t_{1/2}s=102 min), anti IL13-2 (t_{1/2}s=10 min), and anti IL13-1 (t_{1/2}s=1.3 min) are not only separated from the beads coated with the longest t_{1/2} mAb (anti IL13-4), but also closer to each other in the dot plot. The addition of the second color in this chase helps to further separate beads coated with the two longer t_{1/2}s from each other.

Thus, B cells expressing surface-anchored antibodies with fast or slow dissociation constants can be separated by using the various chase conditions of the disclosure to label the B cells and incorporating into sort strategy to enrich for high affinity antibodies.

### Example 3: Separation of B Cells in FACS Experiments Based on Antigen Binding Dissociation Rates

During the standard B cell sorting technology workflow, splenocytes from immunized mice are stained with fluorescently labeled antigen, washed, and then single-cell sorted using flow cytometry. Sort gating is primarily based on selection of IgG and antigen double positive populations and can thus isolate antigen specific B cells which express antibodies with a range of affinities. This process has previously been refined to identify higher affinity antibodies by utilizing monomeric sort reagents and by specifically sorting cells with the highest antigen specific+/IgG+ ratios (“diagonal sorting”).

In this example, the sorting process of B cells was further refined by adding a chase step designed to remove B cells expressing lower affinity antibodies from B cells expressing high affinity antigen specific antibodies. In this improved workflow, the chase step was carried out in the presence of a multimerized and differentially labeled antigen with or without additional unlabeled (“cold”) antigen. AlexaFluor647 (A647) labeled human IL13 was incubated with mouse splenocytes for 30 minutes followed by 2 washes to remove the unbound sort reagent and present only B cells bound to the labeled human IL13. The labeled B cells further underwent a chase step using one of two methods: Multivalent Chase or Combined (cold monovalent chase and hot multivalent) Chase. The Multivalent Chase consisted of pre-clustering Biotin-hIL13 with Streptavidin-PE at a 4 to 1 ratio, leading to a mixture of tetramers, trimers, dimers and some monomers. The Combined Chase consisted of a “cold” (unlabeled) monovalent hIL3 combined with the Multivalent Chase antigen. These chase approaches greatly improved the efficiency of the isolation process for mAbs with ultra-high affinities by reducing the total number of antibodies that need to be cloned, expressed, and screened.

Ultra-high affinity antibodies were isolated against a soluble cytokine target, IL13. Genetically modified mice that have unrearranged human immunoglobulin variable region gene segments at endogenous mouse immunoglobulin loci were generated by methods described in U.S. Pat. No. 8,502,018. The genetically modified mice were immunized with human IL13 in the form of mFc dimer proteins and fusions with antibodies targeting antigen presenting cells. Implementing the chase strategies disclosed herein, a total of 58 antibodies with t_{1/2} longer than 100 minutes were identified during B cell isolation. Over 50% of all antibodies isolated using this method had nanomolar or better affinities. Of these, 47 antibodies had ultra-high affinities, affinities below 1×10⁻¹⁰ (M). This is in sharp contrast to 20% of antibodies with sub-nanomolar affinities that were identified using a traditional sorting strategy without any “chase” steps, yielding only 7 ultra-high affinity antibodies. Among the 47 ultra-high affinity antibodies, 39 were also potent inhibitors of IL13 in bioassays. Therefore, the disclosed B cell sorting strategy offers a significant improvement over the current method for those applications where high affinity antibodies are required to achieve therapeutic efficacy.

After the B cells were sorted from IL13 immunized mice as described, the variable domains for both heavy and light chains were cloned into fully human IgG1 expression constructs and transiently expressed in CHO cells. Antibody containing supernatants were assayed for target specific engagement by Luminex or SPARCL assays. All IL13 binders across all sorting strategies were moved to Biacore and Bioassay screening and the results are shown in Table 2. The numbers shown in Table 2 refer to the number of mice used in the specific sorting strategy, where each mouse could be used across multiple sort strategies. Therefore, the total number of mice sorted overall was 13.

TABLE 2

| | Mice | B Cells | Antibodies | |
|---|---|---|---|---|
| Sort Strategy | Sorted | Collected | Cloned | IL13 Binders (%) |
| No Chase | 12 | 1496 | 637 | 519 (81%) |
| Multimeric Chase | 6 | 675 | 199 | 157 (79%) |
| Cold + Multimeric | 13 | 1907 | 775 | 700 (90%) |
| Chase | | | | |
| TOTAL | 13 | 4078 | 1611 | 1376 (85%) |

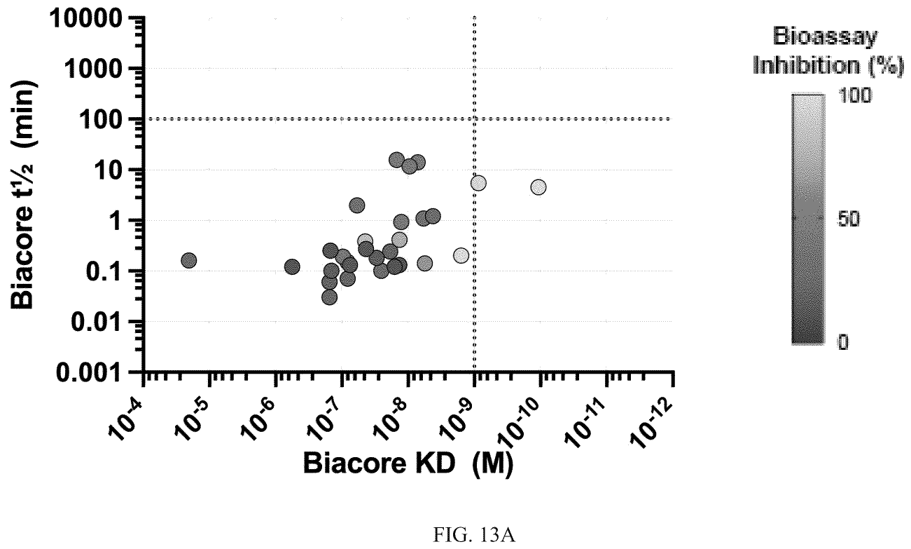

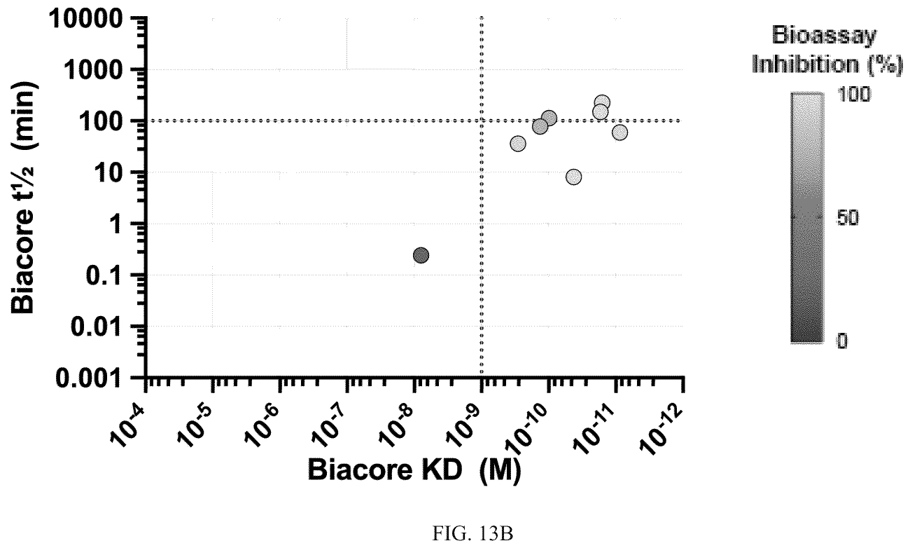

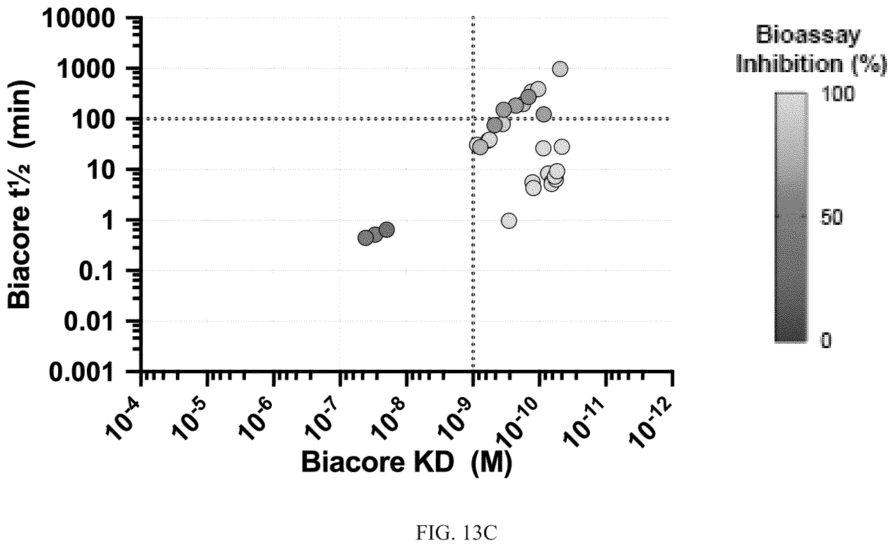

Antibodies from a representative mouse were analyzed by Biacore and results are shown in FIG. 13A-C. In the No Chase condition (FIG. 13A), the antibodies that were isolated were mostly low affinity with short t_{1/2}s. However, the antibodies that were isolated using either of the two chase strategies (FIG. 13B and FIG. 13C) show a marked enrichment for sub-nanomolar affinity antibodies with t_{1/2}s over 100 minutes. In addition to having improved affinity properties, these antibodies were also potent inhibitors in the IL13 inhibition assay. This is seen in FIG. 13B-C designated by the color of the data points where yellow is 100% inhibition.

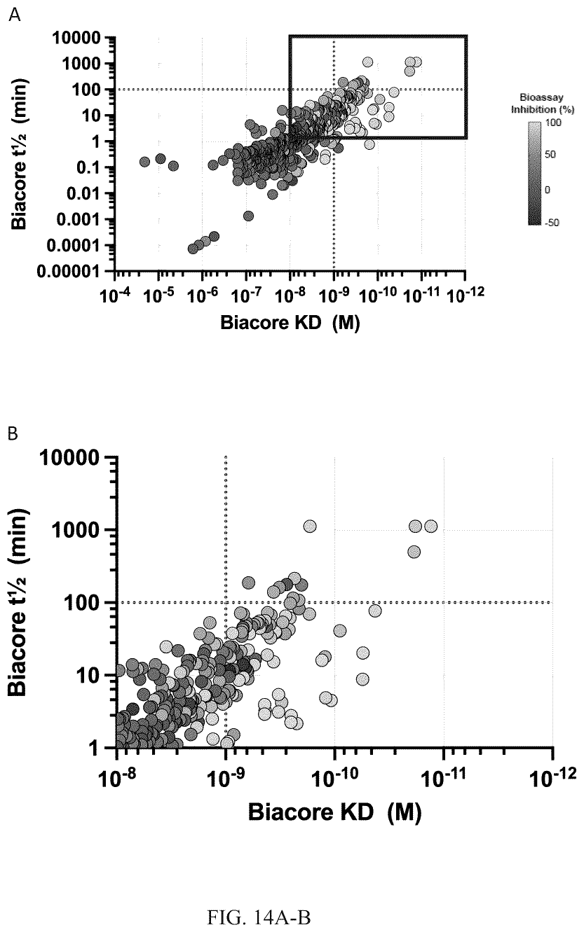

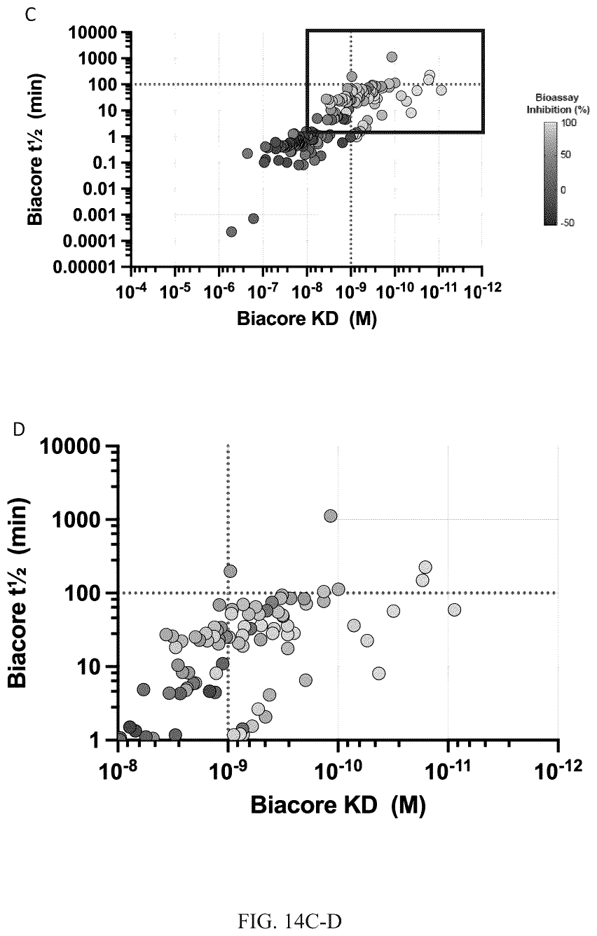

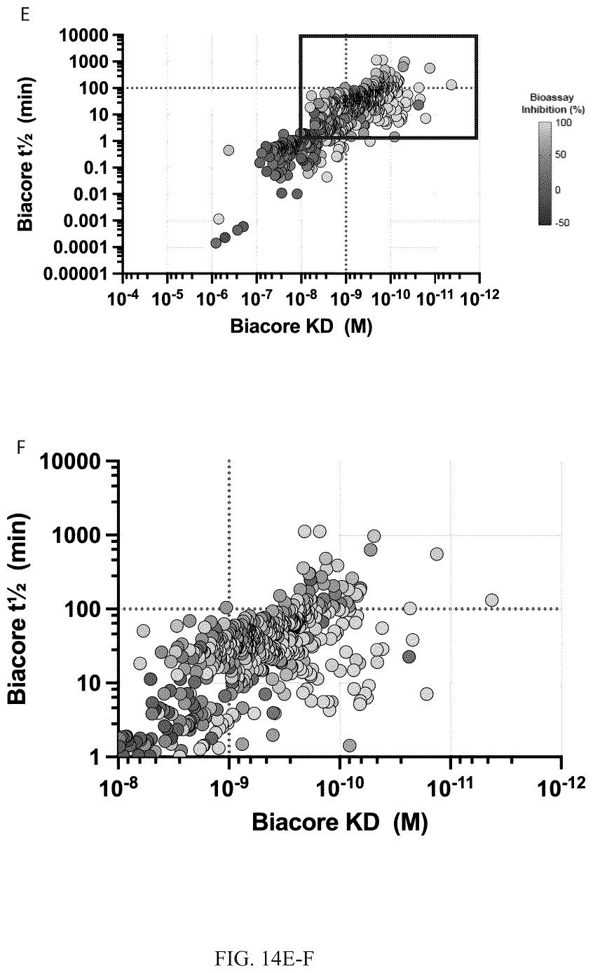

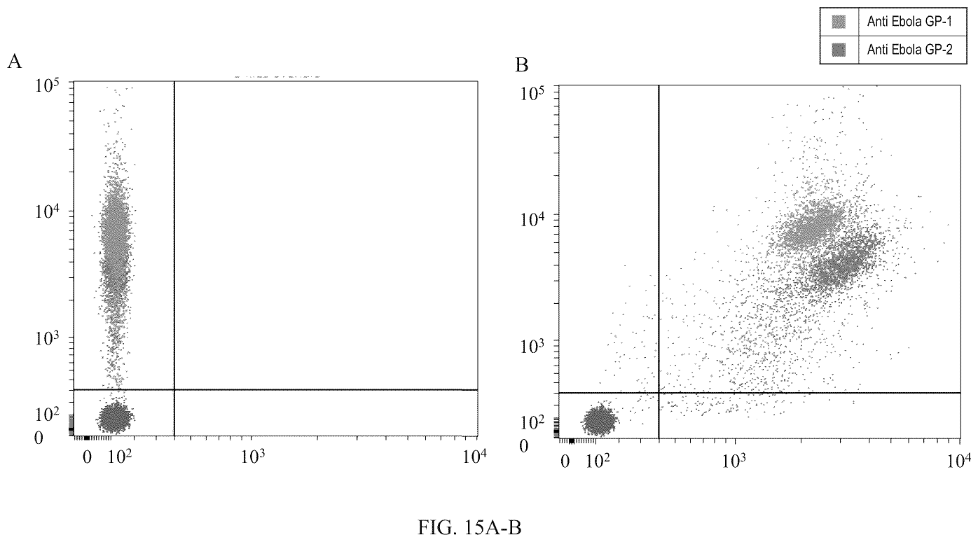

The antibodies analyzed by Biacore from all experimental mice are shown in Table 3. The trends seen in all the mice are consistent with those seen in the individual mouse example of FIG. 13A-C. The zoomed-in views FIGS. 14B, 14D, and 14F focus on the higher affinity antibodies. Along with FIGS. 14A-F, Table 3 shows that a greater than three-fold enrichment in longer t_{1/2} antibodies and greater than two-fold enrichment in the overall K_{D}s was observed. Additionally, as seen in FIGS. 14E and 14F, the high affinity antibodies from the Cold Monovalent+Multivalent Chase strategy also have a propensity for being strong IL13 bioassay blockers (100-200 pM hIL13 was used in bioassay).

Overall, the Cold+Multimeric Chase strategy enriched more than three-fold (2.3% to 7.4%) for antibodies with longer t_{1/2}. Even though IL13 was used in these examples, the chase strategies presented herein can be implemented for other targets where ultra-high affinity antibodies are required.

TABLE 3

| | | | | | Multimeric Chase (157 | | | | Cold + Multimeric Chase | | | |
|---|---|---|---|---|---|---|---|---|---|---|---|---|
| | No Chase (519 Abs) | | | | Abs) | | | | (700 Abs) | | | |
| | t_{1/2} > 100 min: 12 (2.3%) | | | | t_{1/2} > 100 min: 6 (3.8%) | | | | t_{1/2} > 100 min: 52 (7.4%) | | | |
| | Total | | Total | | Total | | Total | | Total | | Total | |
| | Abs | | Inhibitors | | Abs | | Inhibitors | | Abs | | Inhibitors | |
| | (%) | | (%) | | (%) | | (%) | | (%) | | (%) | |
| 1 × 10⁻¹¹ > K_{D} > | 0 | (0.0%) | 0 | (0.0%) | 1 | (0.6%) | 1 | (0.6%) | 1 | (0.1%) | 1 | (0.1%) |
| 1 × 10⁻¹² M | | | | | | | | | | | | |
| 1 × 10⁻¹⁰ > K_{D} > | 7 | (1.3%) | 7 | (1.3%) | 7 | (4.5%) | 6 | (3.8%) | 38 | (5.4%) | 31 | (4.4%) |
| 1 × 10⁻¹¹ M | | | | | | | | | | | | |
| 1 × 10⁻⁹ > K_{D} > | 96 | (18.5%) | 43 | (8.3%) | 44 | (28.0%) | 29 | (18.5%) | 349 | (49.9%) | 218 | (31.1%) |
| 1 × 10⁻¹⁰ M | | | | | | | | | | | | |
| 2.5 × 10⁻⁸ > K_{D} > | 89 | (17.1%) | 1 | (0.20%) | 36 | (22.9%) | 0 | (0.0%) | 60 | (15.0%) | 0 | (0.0%) |
| 1 × 10⁻⁹ M | | | | | | | | | | | | |
| TOTAL | 192 | (36.9%) | 51 | (9.8%) | 88 | (56.1%) | 36 | (22.9%) | 448 | (64.0%) | 250 | 5.7%) |

### Example 4. Proof of Concept: Separation of Antibody-Coated Beads Based on Dissociation Rates Between the Antibodies and the Multimeric Antigen in FACS Experiments

Purpose: To demonstrate the ability to separate antibody-expressing cells based on dissociation rates between the antibodies and the multimeric antigen, Ebola GP trimer protein, using fluorophore labeled multivalent chase method in FACS-based experiments.

In this proof-of-concept experiment, polystyrene microbeads were used in place of antibody-expressing cells. As described in example 1, beads were separately coated with two monoclonal antibodies against Zaire Ebola GP protein with different affinities: anti Ebola GP-1, and anti Ebola GP-2. The dissociation t_{1/2} of the binding between these two antibodies are 1155 minutes for anti Ebola GP-1, and 3 minutes for anti Ebola GP-2, as shown in Table 4.

TABLE 4

| Anti-Ebola Abs | KD (M) | t½ (min) |
|---|---|---|
| anti Ebola GP-1 | 2.28E−10 | 1155 |
| anti Ebola GP-1 | 5.23E−08 | 3 |

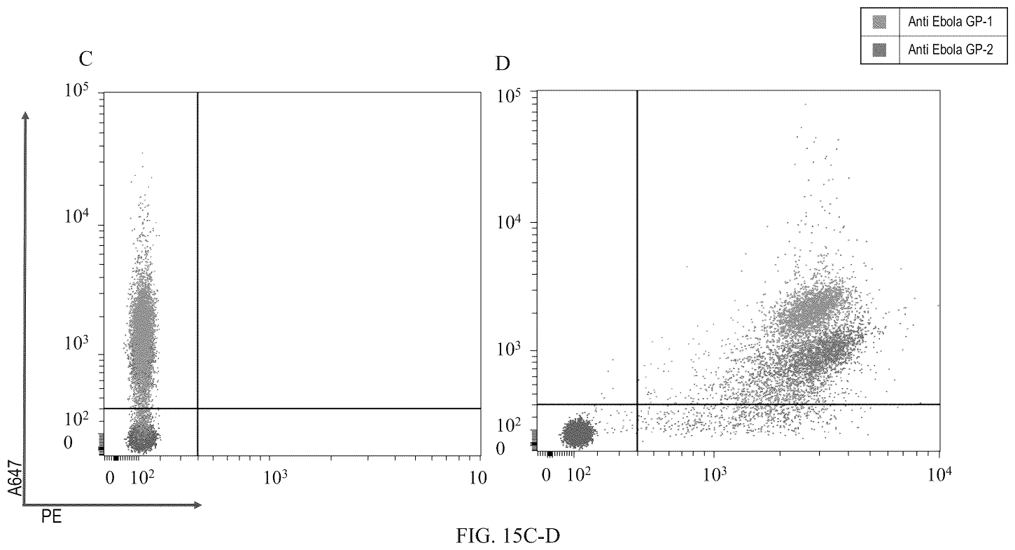

The antibody coated beads were then incubated with A647 conjugated Ebola GP trimer protein at either 5 nM or 0.2 nM for 30 minutes. Unbound A647 conjugated Ebola GP trimer protein was removed through two washes using stain buffer, and the antibody coated beads were further applied with either no-chase condition, or fluorophore labeled multivalent chase condition. Samples are later analyzed by flow cytometry. The results are shown in A647 vs PE 2-color dot plot overlays in FIG. 15.

In samples without chase, no further steps were needed. Samples were pelleted by centrifugation and resuspended in PBS before analyzed by flow cytometry. For beads incubated with A647 conjugated Ebola GP trimer protein at 5 nM as shown in FIG. 15A, A647 MFI for anti Ebola GP-1 coated beads (shaded in green) is 6,335, and for anti Ebola GP-2 coated beads (shaded in purple) is 3,781. Though they stain differently, the overlay showed the two populations are heavily overlapped and thus hard-to-separate in A647 staining (Y-axis). FIG. 15C showed similar results for beads incubated with Ebola GP trimer protein at 0.2 nM. A647 MFI for anti Ebola GP-1 coated beads (shaded in green) is 1,466, and for anti Ebola GP-2 coated beads (shaded in purple) is 908. The two bead populations are heavily overlapped and inseparable in the overlay.

In beads with fluorophore labeled multivalent chase method applied, after the removal of unbound A647 conjugated Ebola GP trimer protein, antibody coated beads were incubated with 20 nM biotin labeled Ebola GP trimer protein pre-bound with 5 nM Phycoerythrin (PE)-streptavidin (SA) for 45 minutes, followed by two washes of PBS before the second incubation of 20 nM biotin labeled Ebola GP trimer protein pre-bound with 5 nM PE-SA for another 45 minutes. After the second incubation, the beads were pelleted by centrifugation, resuspended in PBS, and analyzed by flow cytometry.

Fluorophore labeled multivalent chase experiment results are shown in A647 vs PE 2-color dot plot overlays in FIGS. 15B and 15D. With A647 labeled Ebola GP trimer protein applied at 5 nM in FIG. 15B, when looking only at A647 staining level, both anti Ebola GP-1 and anti Ebola GP-2 coated beads that underwent fluorophore labeled multivalent chase showed the similar differential A647-staining levels as their corresponding antibody-coated beads with no chase shown in FIG. 15A; however, the two antibody-coated populations are pulled away from each other when also take PE staining level into account in the dot plot. Beads shaded in green (t_{1/2}=1155 min, coated with anti Ebola GP-1) showed less PE staining and more A647 staining, which is separated from beads shaded in purple (t_{1/2}=3 min, coated with anti Ebola GP-2) that has more PE staining and less A647 staining. The addition of the second color in this chase helped to separate beads with different t_{1/2}s from each other. Beads coated with the two different antibodies can be separated from each other using fluorophore labeled multivalent chase method based on the dissociation rates in the case for trimer antigen binding.

1. A method for obtaining antibody-producing cells that express antibody molecules exhibiting high binding affinity for an antigen, the method comprising:
(a) contacting a population of antibody-producing cells encompassing cells that express antibody molecules to the antigen on the cell surface, with a first labeled form of the antigen to allow the antigen to bind to the antibody molecules on the cell surface, wherein the antigen of the first labeled form is conjugated to a first detectable label;
(b) washing the cells to remove unbound antigen;
(c) contacting the cells with
(i) an unlabeled form of the antigen,
(ii) a second labeled form of the antigen, or
(iii) the unlabeled form of the antigen and the second labeled form of the antigen;
(d) washing the cells to remove unbound antigen; and
(e) collecting cells that remain bound to the first labeled form of the antigen, thereby obtaining cells expressing high affinity antibody molecules to the antigen.

2. The method of claim 1, wherein the first labeled form of the antigen is at a concentration between 0.001 nM and 1 μM.

3. (canceled)

4. The method of claim 1, wherein the first detectable label is a first fluorescent label.

5. The method of claim 1, wherein the antigen is a protein in a monomeric form.

6. The method of claim 1, wherein the antigen is a protein in a multimeric form.

7. The method of claim 1, wherein the antigen is a protein present in both monomeric and multimeric forms.

8. The method of claim 1, wherein the first labeled form of the antigen is a monovalent form of the antigen.

9. The method of claim 1, wherein the unlabeled form of the antigen is a monovalent form of the antigen.

10. The method of claim 1, wherein the unlabeled form of the antigen is a multivalent form of the antigen.

11. The method of claim 10, wherein the multivalent form of the antigen is provided by a multivalent molecule to which the antigen is bound or linked.

12. The method of claim 11, wherein the multivalent molecule is selected from a streptavidin multimer, a dimer of an immunoglobulin Fc fragment, or a trimer of a trimerization molecule.

13. The method of claim 1, wherein the second labeled form of the antigen is a monovalent form of the antigen.

14. The method according to claim 1, wherein the second labeled form of the antigen is a multivalent form of the antigen.

15. The method of claim 14, wherein the multivalent form of the antigen is provided by a multivalent molecule to which the antigen is bound.

16. The method of claim 15, wherein the multivalent molecule is a streptavidin multimer, a dimer of an immunoglobulin Fc fragment, or a trimer of a trimerization molecule.

17. The method of claim 14, wherein the multivalent form of the antigen is labeled with a second detectable label.

18. The method of claim 17, wherein the second detectable label is a second fluorescent label.

19. The method of claim 8, wherein in step (c) the cells are contacted with the unlabeled form of the antigen.

20. The method of claim 19, wherein the unlabeled form of the antigen is a monovalent form of the antigen.

21. The method of claim 19, wherein the antigen in the unlabeled form is at least 2 to 4 fold in molar ratio relative to the first labeled form of the antigen used in step (a).

22. The method of claim 8, wherein in step (c) the cells are contacted with the second labeled form of the antigen.

23. The method of claim 22, wherein the second labeled form of the antigen is a multivalent form of the antigen.

24. The method of claim 22, wherein the antigen in the second labeled form is at least 2 to 4-fold in molar ratio relative to the first labeled form of the antigen used in step (a).

25. (canceled)

26. The method of claim 8, wherein in step (c) the cells are contacted with an unlabeled form of the antigen and a second labeled form of the antigen.

27. The method of claim 26, wherein the unlabeled form is a monovalent form of the antigen, and the second labeled form of the antigen is a multivalent form of the antigen.

28. The method of claim 26, wherein the antigen is a monomeric protein, the unlabeled form is a monovalent form of the antigen, and the second labeled form of the antigen is a multivalent form of the antigen.

29. The method of claim 26, wherein the antigen is a multimeric protein, the unlabeled form is a monovalent form of the antigen, and the second labeled form of the antigen is a multivalent form of the antigen.

30. The method of claim 26, wherein the antigen is a protein present in both a monomeric and a multimeric form, the unlabeled form is a monovalent form of the antigen, and the second labeled form of the antigen is a multivalent form of the antigen.

31. The method of claim 26, wherein the cells are contacted with the unlabeled form of the antigen and the second labeled form of the antigen at the same time.

32. The method of claim 26, wherein the unlabeled form of the antigen is at least 2 to 4-fold in molar ratio relative to the first labeled form of the antigen used in step (a).

33. The method of claim 1, wherein the first detectable label is a fluorescent label, and wherein fluorescence-activated cell sorting is used to collect cells that remain bound to the first labeled form of the antigen.

34. The method of claim 1,
wherein in step (c), the cells are contacted with the second labeled form of the antigen, or with the unlabeled form of the antigen and the second labeled form of the antigen,
wherein the second labeled form of the antigen is conjugated with a second detectable label,
wherein the first detectable label is a first fluorescent label, and the second detectable label is a second fluorescent label that differentiates from the first fluorescent label, and
wherein two-dimensional fluorescence-activated cell sorting is used to collect cells that remain bound to the first labeled form of the antigen.

35. The method of claim 1, wherein the antibody producing cells are primary antibody producing cells, yeast, or immortalized mammalian cells which produce antibody molecules on the cell surface.

36. The method according to claim 35, wherein the primary antibody-producing cells are obtained from spleen, lymph node, peripheral blood and/or bone marrow.

37. The method according to claim 35, wherein the immortalized mammalian cells which produce antibody molecules are selected from Chinese hamster ovary (CHO) cells and hybridoma cells.

38. The method of claim 1, wherein the high affinity is in the range of from about 0.1 pM to about 25 nM (KD).

39. The method of claim 38, wherein the high affinity is less than about 10 nM (KD).

40. The method of claim 1, further comprising: isolating antibody-encoding nucleic acids from the cells collected in step (e).

41.-43. (canceled)
